SEQUENCE LISTING (62 sequences)

SEQ ID NO: 1 - DNA, 21 bp, Artificial Sequence
  shRNA-147 target sequence
aagtgaagaatacgatcaagt

SEQ ID NO: 2 - DNA, 29 bp, Artificial Sequence
  shRNA-1234 target sequence
ggtcacaagattccacattaattgggaag

SEQ ID NO: 3 - DNA, 29 bp, Artificial Sequence
  shRNA-4 target sequence
gcaaaggttcaggtgaacaatgtagtggt

SEQ ID NO: 4 - DNA, 21 bp, Artificial Sequence
  shRNA-238 target sequence
aatccaggactcattccagat

SEQ ID NO: 5 - DNA, 20 bp, Artificial Sequence
  Primer to analyze the Oct4 promoter region forward
tgaactgtggtggagagtgc

SEQ ID NO: 6 - DNA, 20 bp, Artificial Sequence
  Primer to analyze the Oct4 promoter region reverse
aggaagggctaggacgagag

SEQ ID NO: 7 - DNA, 26 bp, Artificial Sequence
  Negative control primer forward
ttttcagttcacacatataaagcaga

SEQ ID NO: 8 - DNA, 22 bp, Artificial Sequence
  Negative control primer reverse
tgttgttgttgttgcttcactg

SEQ ID NO: 9 - DNA, 44 bp, Artificial Sequence
  ASF1A cloning forward
ggcgctttgtttaaacccggcaccatggcaaaggttcaggtgaa

SEQ ID NO: 10 - DNA, 39 bp, Artificial Sequence
  ASF1A cloning reverse
ggccgaagggtttaaaccctcacatgcagtccatgtggg

SEQ ID NO: 11 - DNA, 20 bp, Artificial Sequence
  POU5F1 endog forward
cctcacttcactgcactgta

SEQ ID NO: 12 - DNA, 20 bp, Artificial Sequence
  POU5F1 endog reverse
caggttttctttccctagct

SEQ ID NO: 13 - DNA, 23 bp, Artificial Sequence
  POU5F1 transgene forward
ccccagggccccattttggtacc

SEQ ID NO: 14 - DNA, 25 bp, Artificial Sequence
  POU5F1 transgene reverse
cttccctccaaccagttgccccaac

SEQ ID NO: 15 - DNA, 20 bp, Artificial Sequence
  POU5F1 forward
ggttctatttgggaaggtat

SEQ ID NO: 16 - DNA, 19 bp, Artificial Sequence
  POU5F1 reverse
catgttcttgaagctaagc

SEQ ID NO: 17 - DNA, 21 bp, Artificial Sequence
  ASF1A transgene forward
tttaaacccggcaccatggca

SEQ ID NO: 18 - DNA, 20 bp, Artificial Sequence
  ASF1A transgene reverse
gcatctgcatctggaatgag

SEQ ID NO: 19 - DNA, 19 bp, Artificial Sequence
  ASF1A forward
ccgcaggaaggcatatgtt

SEQ ID NO: 20 - DNA, 20 bp, Artificial Sequence
  ASF1A reverse
gcatctgcatctggaatgag

SEQ ID NO: 21 - DNA, 20 bp, Artificial Sequence
  actin forward
tgaagtgtgacgtggacatc

SEQ ID NO: 22 - DNA, 20 bp, Artificial Sequence
  actin reverse
ggaggagcaatgatcttgat

SEQ ID NO: 23 - DNA, 20 bp, Artificial Sequence
  hTERT forward
tgtgcaccaacatctacaag

SEQ ID NO: 24 - DNA, 20 bp, Artificial Sequence
  hTERT reverse
gcgttcttggctttcaggat

SEQ ID NO: 25 - DNA, 20 bp, Artificial Sequence
  hGDF3 forward
aaatgtttgtgttgcggtca

SEQ ID NO: 26 - DNA, 20 bp, Artificial Sequence
  hGDF3 reverse
tctggcacaggtgtcttcag

SEQ ID NO: 27 - DNA, 19 bp, Artificial Sequence
  SOX2 forward
cccagcagacttcacatgt

SEQ ID NO: 28 - DNA, 20 bp, Artificial Sequence
  SOX2 reverse
cctcccatttccctcgtttt

SEQ ID NO: 29 - DNA, 20 bp, Artificial Sequence
  KLF4 forward
gatgaactgaccaggcacta

SEQ ID NO: 30 - DNA, 20 bp, Artificial Sequence
  KLF4 reverse
gtgggtcatatccactgtct

SEQ ID NO: 31 - DNA, 20 bp, Artificial Sequence
  DNMT3B forward
ataagtcgaaggtgcgtcgt

SEQ ID NO: 32 - DNA, 20 bp, Artificial Sequence
  DNMT3B reverse
ggcaacatctgaagccattt

SEQ ID NO: 33 - DNA, 20 bp, Artificial Sequence
  NANOG forward
tacctcagcctccagcagat

SEQ ID NO: 34 - DNA, 20 bp, Artificial Sequence
  NANOG reverse
tctggaaccaggtcttcacc

SEQ ID NO: 35 - DNA, 22 bp, Artificial Sequence
  GAPDH forward
atggaaatcccatcaccatctt

SEQ ID NO: 36 - DNA, 18 bp, Artificial Sequence
  GAPDH reverse
cggcccacttgattttgg

SEQ ID NO: 37 - DNA, 24 bp, Artificial Sequence
  REX1 forward
cccacagtccatccttacagagtt

SEQ ID NO: 38 - DNA, 18 bp, Artificial Sequence
  REX1 reverse
gggactttgcccccaaac

SEQ ID NO: 39 - DNA, 20 bp, Artificial Sequence
  RUNX1 forward
ccctaggggatgttccagat

SEQ ID NO: 40 - DNA, 20 bp, Artificial Sequence
  RUNX1 reverse
tgaagcttttccctcttcca

SEQ ID NO: 41 - DNA, 24 bp, Artificial Sequence
  BRACHYURY forward
accaccgctggaaatatgtgaacg

SEQ ID NO: 42 - DNA, 24 bp, Artificial Sequence
  BRACHYURY reverse
aactctcacgatgtgaatccgagg

SEQ ID NO: 43 - DNA, 21 bp, Artificial Sequence
  NESTIN forward
cagcgttggaacagaggttgg

SEQ ID NO: 44 - DNA, 23 bp, Artificial Sequence
  NESTIN reverse
tggcacaggtgtctcaagggtag

SEQ ID NO: 45 - DNA, 20 bp, Artificial Sequence
  AFP forward
agcttggtggtggatgaaac

SEQ ID NO: 46 - DNA, 20 bp, Artificial Sequence
  AFP reverse
ccctcttcagcaaagcagac

SEQ ID NO: 47 - DNA, 26 bp, Artificial Sequence
  NCAM forward
atggaaactctattaaagtgaacctg

SEQ ID NO: 48 - DNA, 25 bp, Artificial Sequence
  NCAM reverse
tagacctcatactcagcattccagt

SEQ ID NO: 49 - DNA, 20 bp, Artificial Sequence
  PAX6 forward
cggagtgaatcagctcggtg

SEQ ID NO: 50 - DNA, 23 bp, Artificial Sequence
  PAX6 reverse
ccgcttatactgggctattttgc

SEQ ID NO: 51 - DNA, 18 bp, Artificial Sequence
  GATA4 forward
ctctacatgaagctccac

SEQ ID NO: 52 - DNA, 18 bp, Artificial Sequence
  GATA4 reverse
ctgctggtgtcttagatt

SEQ ID NO: 53 - DNA, 24 bp, Artificial Sequence
  ChIP NANOGF forward
gaaagacatgacaaatcaccagac

SEQ ID NO: 54 - DNA, 24 bp, Artificial Sequence
  ChIP NANOGF reverse
caactagctccattttcctctttc

SEQ ID NO: 55 - DNA, 24 bp, Artificial Sequence
  ChIP SOX2 forward
cggttgaatgaagacagtctagtg

SEQ ID NO: 56 - DNA, 24 bp, Artificial Sequence
  ChIP SOX2 reverse
cgactagaagttaggagacccaaa

SEQ ID NO: 57 - DNA, 23 bp, Artificial Sequence
  ChIP OCt4 forward
ttacttaagcgacagaggtcagc

SEQ ID NO: 58 - DNA, 24 bp, Artificial Sequence
  ChIP OCt4 reverse
tggtctagtgcttgattctgtttg

SEQ ID NO: 59 - DNA, 24 bp, Artificial Sequence
  ChIP KRTHA4 forward
taggtatactcccatccattccat

SEQ ID NO: 60 - DNA, 24 bp, Artificial Sequence
  ChIP KRTHA4 reverse
tagcagaaactcaacctgtattcg

SEQ ID NO: 61 - DNA, 20 bp, Artificial Sequence
  ChIP intergenic forward
aatgagtgggctcatggaaa

SEQ ID NO: 62 - DNA, 20 bp, Artificial Sequence
  ChIP intergenic reverse
tctggatgcagcatttgtgt

(19) United States
(12) Patent Application Publication
(10) Pub. No.: US 2017/0211091 A1
(43) Pub. Date: Jul. 27, 2017

(54) METHODS FOR GENERATING INDUCED PLURIPOTENT STEM CELLS

(71) Applicants: FUNDACIÓN PÚBLICA ANDALUZA PROGRESO Y SALUD, Sevilla (ES); MICHIGAN STATE UNIVERSITY, East Lansing, MI (US); INSTITUTO DE SALUD CARLOS III, Madrid (ES)

(72) Inventors: Jose B. Cibelli, East Lansing, MI (US); María Elena Gonzalez-Muñoz, Sevilla (ES)

(73) Assignees: FUNDACIÓN PÚBLICA ANDALUZA PROGRESO Y SALUD, Sevilla (ES); MICHIGAN STATE UNIVERSITY, East Lansing, MI (US); INSTITUTO DE SALUD CARLOS III, Madrid (ES)

(21) Appl. No.: 15/326,863

(22) PCT Filed: Jul. 16, 2015
(86) PCT No.: PCT/EP2015/066279

§ 371 Date: Jan. 17, 2017

Related U.S. Application Data
(60) Provisional application No. 62/025,279, filed on Jul. 16, 2014.

(51) Int. Cl. C12N 15/86

(52) U.S. Cl. CPC C12N 15/86; C12N 2740/10043

(57) ABSTRACT

Provided herein are methods and compositions for inducing a somatic cell to acquire a less differentiated phenotype and for generating induced pluripotent stem cells (i PS cells) by inducing expression of ASF1A in the cell and/or by contacting the cell with GDF9. Also provided herein are compositions and methods for treating and/or diagnosing cancer and for identifying agents useful in the treatment and/or diagnosis of cancer.

### BACKGROUND

Induced expression of certain sets of proteins (referred to as “reprogramming factors”) in a somatic cell can cause the cell to change its phenotype from a differentiated state to an undifferentiated state, becoming an “induced pluripotent stem cell” (“iPS cell” or “iPSC”). Takahashi and Yamanaka, Cell 126:663-676 (2006). Like embryonic stem cells (“ES cells”), iPS cells are able to give rise to every other cell type in the body. Accordingly, such cells hold great promise in the field of regenerative medicine. What is more, since no human embryos are destroyed in production of iPS cells, many of the ethical concerns surrounding the use of conventional human ES cells are not applicable to iPS cells. Additionally, since iPS cells can be derived directly from adult tissue, iPS cells can be made that genetically match a patient. Use of such genetically matched cells in regenerative medicine greatly reduces the risk of immune rejection compared to the use of traditional ES cells. Genetically matched iPS cells and differentiated cells generated from such iPS cells can also be used to screen potential therapeutic agents to determine the agent's likely efficacy on the individual from whom the iPS cell was made. Such cells therefore provide a valuable tool for personalized medicine and drug discovery.

To date, the use of iPS cells has been limited by the methods available for their generation. iPS cells are traditionally made by introducing a set of pluripotency associated genes, referred to as reprogramming factors, into a somatic cell. The traditional set of reprogramming factors, referred to as Yamanaka factors, include the genes encoding the transcription factors OCT3/4, SOX2, c-MYC and KLF4. However, iPS cell derivation using this traditional approach is inefficient, with only about 0.01%-0.1% of the transfected cells becoming pluripotent. What is more, two of the Yamanaka factors, c-MYC and KLF4, are known oncogenes. Indeed, 20% of chimeric mice derived from iPS cells acquire cancer. The concern over the oncogenic potential of traditionally derived iPS cells has limited their therapeutic development.

Accordingly, there is a great need for improved compositions and methods for the generation of iPS cells.

### SUMMARY

In certain aspects, provided herein are methods (e.g., in vitro methods) of inducing a somatic cell (e.g., a mammalian cell, such as a human cell) to acquire a less differentiated phenotype and for the generation of iPS cells. In some embodiments, the method includes a step of inducing expression of ASF1A in a somatic cell. In some embodiments, the method includes a step of contacting a somatic cell with GDF9. In some embodiments, the method includes a step of inducing expression of one or more reprogramming factors in the cell (e.g., OCT3/4, NANOG, SOX1, SOX2, SOX3, SOX15, SOX18, DNMT3B, c-MYC, N-MYC, L-MYC, KLF1, KLF2, KLF4, KLF5, LIN28 and/or GLIS1). In some embodiments, expression of both ASF1A and OCT3/4 is induced in the cell. In some embodiments, the cell is contacted with GDF9 after expression of one or more reprogramming factors is induced in the cell (e.g., after ASF1A and OCT3/4 is expressed in the cell). In some embodiments, the somatic cell is a fibroblast.

In some embodiments, the expression of ASF1A and/or one or more reprogramming factors is induced in the cell by contacting the cell with one or more expression vectors encoding the reprogramming factor(s). In some embodiments, the expression vector is a retroviral vector (e.g., a self-inactivating retroviral vector). In some embodiments the expression vector is a lentiviral vector, an adenovirus vector, a plasmid vector and/or linear DNA. In some embodiments, ASF1A and/or reprogramming factor protein is directly introduced into the cell in combination with and/or instead of an expression vector.

In some embodiments, expression of ASF1A and/or one or more reprogramming factors is induced by contacting the cell with an agent that induces expression of the reprogramming factor in the cell. In some embodiments, the cell is contacted with one or more agents that enhance the dedifferentiation process (e.g., a histone deacetylase inhibitor, such as valproic acid, a histone methyl transferase inhibitor, such as BIX-01294, an ALK5 inhibitor such as SB431412, a MEK inhibitor such as PD0325901).

In some embodiments, the methods provided herein include culturing the cell under conditions whereby the cell acquires a less differentiated phenotype (e.g., become an iPS cell). In some embodiments the cell is cultured in human ES cell medium. In some embodiments, the human ES cell medium includes GDF9 for at least a portion of the time the cell is being cultured.

In certain aspects, provided herein is a method (e.g., an in vitro method) of determining whether a test agent is an agent useful for inducing a cell (e.g., a human cell, such as a human fibroblast cell) to acquire a less differentiated phenotype and/or for generating iPS cells. In some embodiments, the method includes contacting a cell or cell extract with the test agent. In some embodiments, the method includes detecting the expression or activity of ASF1A in the cell or cell extract. In some embodiments, a test agent that increases the expression and/or activity of ASF1A (e.g., compared to a cell or cell extract that has not been contacted with an agent and/or compared to a cell or cell extract that has been contacted with a control agent) is an agent useful for inducing a cell to acquire a less differentiated phenotype and/or for generating iPS cells. In some embodiments, the expression of ASF1A is detected in the cell by detecting ASF1A mRNA level or ASF1A protein level. In some embodiments, the activity of ASF1A is detected in the cell or cell extract by detecting H3K56 acetylation.

In certain aspects, provided herein is a method of treating cancer in a subject. In some embodiment, the method includes administering to the subject an agent that inhibits the activity or expression of ASF1A. In some embodiments, the agent is a small molecule, a polypeptide or inhibitory nucleic acid. In some embodiments, the agent is a small molecule that inhibits ASF1A activity. In some embodiments, the agent is an inhibitory nucleic acid specific for an mRNA that encodes ASF1A (e.g., a siRNA, a shRNA, or an antisense RNA molecule or a nucleic acid that encodes a siRNA, a shRNA, and/or an antisense RNA molecule).

In certain aspects, provided herein is a method (e.g., an in vitro method) of determining whether a test agent is a candidate therapeutic agent for the treatment of cancer. In some embodiments, the method includes contacting a cell or cell extract with the test agent. In some embodiments, the method includes detecting the expression or activity of ASF1A in the cell or cell extract. In some embodiments, a test agent that decreases the expression or activity of ASF1A (e.g., compared to a cell or cell extract that has not been contacted with an agent and/or compared to a cell or cell extract that has been contacted with a control agent) is a candidate therapeutic agent for treating cancer. In some embodiments, the expression of ASF1A is detected in the cell by detecting ASF1A mRNA level or ASF1A protein level. In some embodiments, the activity of ASF1A is detected in the cell or cell extract by detecting H3K56 acetylation.

In some aspects, provided herein is a method (e.g., an in vitro method) of determining whether a subject has cancer and/or is at increased risk for developing cancer. In some aspects, provided herein is a method (e.g., an in vitro method) of determining whether an agent that inhibits ASF1A activity and/or expression will be effective in the treatment of a tumor. In some embodiments, the method includes the step of obtaining a biological sample from the subject (e.g., a tumor sample). In some embodiments, the method includes detecting the expression or activity of ASF1A in a sample from the subject. In some embodiments, elevated expression or activity in ASF1A in the sample (e.g., compared to a control sample of the same type) indicates that the subject has cancer, is at an increased risk for developing cancer and/or that an agent of that inhibits ASF1A activity and/or expression will be effective in the treatment of a tumor in the subject. In some embodiments, the expression of ASF1A is detected in the sample by detecting ASF1A mRNA level or ASF1A protein level. In some embodiments, the activity of ASF1A is detected in the sample by detecting H3K56 acetylation.

In some further aspects, provided herein is a dedifferentiated somatic cell obtained or obtainable according to any of the methods (e.g., in vitro methods) of inducing a somatic cell (e.g., a mammalian cell, such as a human cell) to acquire a less differentiated phenotype and for the generation of iPS cells described herein.

In still some further aspects, provided herein is a dedifferentiated somatic cell characterized by an increased expression and/or activity of ASF1A in comparison to a somatic cell that has not been contacted with an agent capable of increasing the expression and/or activity of ASF1A. Also provided herein is a dedifferentiated somatic cell characterized by an increased expression and/or activity of ASF1A and OCT3/4 in comparison to a somatic cell that has not been contacted with one or more agents capable of increasing the expression and/or activity of ASF1A and OCT3/4. Further provided herein is an induced pluripotent stem (iPS) cell characterized by an increased expression and/or activity of ASF1A in comparison to a somatic cell that has not been contacted with an agent capable of increasing the expression and/or activity of ASF1A, wherein said iPS cell is further characterized by not having an induced expression of oncogenes c-MYC or KLF4. Still further provided herein is an induced pluripotent stem (iPS) cell characterized by an increased expression and/or activity of ASF1A and OCT3/4 in comparison to a somatic cell that has not been contacted with one or more agents capable of increasing the expression and/or activity of ASF1A and OCT3/4, wherein said iPS cell is further characterized by not having an induced expression of oncogenes c-MYC or KLF4.

In some further aspects, provided herein is a cell population comprising a cell as defined in the precedent paragraphs. Preferably, provided herein is a substantially pure population comprising a cell as defined in any of the precedent paragraphs, wherein the term substantially pure is understood as the population comprising a percentage of the cell as defined in any of claims 21 to 26 of at least 80%, preferably 85%, more preferably 90%, 95%, 96%, 97%, 98%, 99% over the total number of cells of the population.

In some further aspects, provided herein is a pharmaceutical composition comprising a cell as defined in any of the precedent paragraphs or the cell population as defined in any of the precedent paragraphs, further comprising a pharmaceutically acceptable carrier.

Finally, still further aspects of the invention provide the cell or the cell population as defined in any of the precedent paragraphs, for use in therapy and for use in a cell therapy method, in particular for use in tissue and/or organ repair and regeneration.

### BRIEF DESCRIPTION OF THE DRAWINGS

FIG. 1 is a Schematic picture of an exemplary somatic cell reprogramming method.

FIG. 2 shows the role of ASF1A during cellular reprogramming. A. H9 hESCs were cultured under conditions to promote spontaneous differentiation. ASF1A expression decreases as pluripotent cells differentiate. Quantitative RT-PCR data for genes characteristic of undifferentiated stem cells was performed as indicated on mRNA collected at days 0, 1, 2, 7 and 12 during differentiation. Mean values (n=3)±SEM are plotted, indicating expression of the specific gene normalized to GAPDH/ACTIN relative to the expression on day 12, which was arbitrarily assigned a value of 0, in a logarithmic scale (1 unit means 10 fold change). B. In the absence of ASF1A, somatic cells cannot reprogram into pluripotent cells when using the Yamanaka factors. 72 hours after hADFs lentiviral transduction with GFP, ASF1A or two different shRNAs against ASF1A, hADFs were transduced with retroviral supernatants encoding OSKM factors for reprogramming. Graph shows number of Tra-1-60⁺ colonies derived from 100,000 cells after OSKM overexpression in GFP (control), ASF1A or the shRNAs 147 or 1234 expressing cells. Data correspond to the average of 3 independent experiments done in duplicate ±SEM, ***P>0.01 compared to control OSKM GFP-expressing fibroblasts. C. Downregulation of ASF1A in H9-hESCs significantly decreases the expression of pluripotency-related genes. qRT-PCR data for ASF1A expression on mRNA collected from H9-hESC cells expressing a lentiviral vector encoding GFP or two different shRNAs against ASF1A (sh147 and sh1234). Mean values (n=3)±SEM are plotted indicating expression of the specific gene normalized to GAPDH/Actin relative to the expression of H9-hES-GFP, which was arbitrarily assigned a value of 0, in a logarithmic scale. Data correspond to the average of 3 independent experiments done in duplicate, ***P>0.001, **>0.05, *>0.01 compared to H9-hESC-GFP.

FIG. 3 shows ASF1A expression during differentiation. A. Immunochemistry analysis of ASF1A expression of H9 hESCs on day 0 and day 12 after spontaneous differentiation using specific anti-ASF1A antibody B. Quantitative RT-PCR data for ASF1A expression of H9 ESCs (hESCs), iPSCs derived using OSKM combination (OSKM-iPSC) and human adult dermal fibroblasts (hADF). Mean values (n=3)±SEM are plotted indicating expression of ASF1A normalized to GAPDH/Actin in a logarithmic scale relative to a hADF sample which was arbitrarily assigned a value of 0. Data correspond to the average of 3 independent experiments performed in duplicate.

FIG. 4 shows ASF1A lentiviral overexpression significantly increases core-pluripotency related genes when overexpressed in (A) human adult dermal fibroblasts (hADF) and (B) H9-ESCs. Control cells were transduced with identical GFP-encoding lentiviral vector. qRTPCRs chart values indicate expression of the specific gene normalized to GAPDH/Actin in Type or paste caption here. a logarithmic scale relative to hADF-GFP sample which was arbitrarily assigned a value of 0. Data correspond to the average of 3 independent experiments done in duplicate±SEM ***P>0.001, **>0.05, *>0.01 Statistics values refer to hADF-GFP.

FIG. 5 shows ASF1A overexpression effect on the H9-ESC differentiation expression pattern. H9-ESCs transduced with a lentiviral vector overexpressing ASF1A (A) delay their onset of core-pluripotency genes downregulation when induced to spontaneously differentiate and (B) delay the onset of upregulation of differentiated related genes when culture. mRNA was collected at days 0, 2, 5 and 7 of spontaneous differentiated H9-ESCs transduced with identical lentiviral vector overexpressing GFP were used as control. All qRT-PCRs chart values indicate expression of the specific gene normalized to GAPDH/Actin in a logarithmic scale compared to H9-hESC-GFP at the different days of differentiation, which was arbitrarily assigned a value of 0. Data correspond to the average of 3 independent experiments done in duplicate, ***P>0.001, **>0.05,*>0.01

FIG. 6 shows ASF1A shRNA efficiency and effect on hADF. ASF1A expression was downregulated using lentiviral vector pLenti-shRNA-GFP encoding four different shRNA for ASF1A. qRT-PCR data for ASF1A expression on mRNA collected from hADFs expressing GFP and scrambled control shRNA (sh-control) or different shRNAs against ASF1A (sh-4, 147, 238 and 1234) 4 days after lentiviral transduction. Mean values (n=3)±SEM are plotted. Chart values indicate expression of ASF1A normalized to GAPDH/Actin in a logarithmic scale relative to sh-control sample which was arbitrarily assigned a value of 0. Data correspond to the average of 3 independent experiments done in duplicate. B. ASF1A downregulation does not affect hADF proliferation rate. 20.000 hADFs were seeded 4 days after transduction with shRNA-147 or sh-1234 constructors. Cells were recovered 2, 4, 5 and 7 days after to measure cellular DNA content via fluorescent dye binding (Cyquant).

FIG. 7 shows downregulation of ASF1A in H9-hESCs significantly decreases the expression of pluripotency-related genes. After ASF1A downregulation, pluripotency related proteins expression also decrease in H9-hESC. Immunochemistry analysis of pluripotent markers (NANOG, SSEA4, TRA-1-60) and ASF1A on hESCs overexpressing GFP (control) or after downregulation of ASF1A using shRNA-1234 two days after bFGF removal from the culture media.

FIG. 8 shows the pluripotent Gene Expression Pattern in hADF after overexpression of ASF1A+KLF4, ASF1A+SOX2, ASF1A+OCT4 and ASF1A+Yamanaka factors. hADF were seeded at 100.000 cells/well and infected with retroviral supernatants encoding each of the single OSK factors (pMX-OCT4, pMX-Sox2 or pMX-KLF4 or OSKM plus ASF1A (pMX-ASF1A) in the presence of 4 μg/ml polybrene. One week after transduction, mRNA was used for qRT-PCR analysis of pluripotent markers (endogenous OCT4, NANOG, SOX2, DNMT3B and GDF3). Mean values (n=3)±SEM are plotted indicating expression of the specific gene normalized to GAPDH/Actin relative to hADF-GFP expression, which was arbitrarily assigned a value of 0, in a logarithmic scale FIG. 9 shows oocyte factors ASF1A and GDF9, and OCT4 (AO9) are sufficient to reprogram somatic cells into pluripotent cells. A. Average of the number of fully reprogrammed iPSC lines derived from 107 transduced hADFs with the different factor combinations: OSKM, OSKM plus NANOG and LIN28 (OSKMNL), OSKM plus ASF1A or GDF9 and ASF1A after GDF9 stimulation (AO9). Mean values (n=3)±SEM are plotted. Fully reprogrammed colonies where considered those that showed all pluripotent markers analyzed in FIG. 13B-C during at least 10 passages. B. Immunocytochemistry analysis of pluripotent markers on AO9 iPSC colonies. Each row shows double staining for the specific colony shown in bright field panel.

FIG. 10 is a table listing oocyte-specific factors screened for their reprogramming capacity.

FIG. 11 shows the morphology and incipient retroviral silencing in colonies emerging in AO9 reprogramming of human dermal fibroblasts 3-4 weeks after transduction. hADF were seeded at 100,000 cells/well and infected with retroviral supernatants encoding each of the single factors (pMXs-OCT4 and pMX-ASF1A-GFP) followed by GDF9 treatment as explain in the Methods section. Fluorescent and bright field pictures were taken 5 days after transduction (upper panel) and when first colonies appear (lower panel).

FIG. 12 shows high-resolution G-banded karyotypes of (A) AO9-iPSC fully reprogrammed, (B) hADF and (C) OSKM-ASF1A iPSCs showing normal karyotype.

FIG. 13 shows ASF1A, OCT4 and GDF9 (AO9) in combination is sufficient for reprogramming hADF to pluripotency. A. qRT-PCR data for genes characteristic of pluripotent cells was performed as indicated on mRNA collected from hADF, H9 hESCs and iPSCs obtained overexpressing ASF1A, OCT4 in the presence of GDF9 (AO9-iPSC). Values indicate expression of the specific gene normalized to GAPDH/Actin in a logarithmic scale relative to hADF sample which was arbitrarily assigned a value of 0. Data correspond to the average of 3 independent experiments done in duplicate. B. Expression array data analysis of similarities between H9-ESCs and AO9-iPSCs (three independent lines AO9-iPSCa, b and c) compared to adult human dermal fibroblasts (hADF). Dendogram and heatmap based on genes up- or downregulated 10-fold or greater versus dermal fibroblasts to visualize similarly expressed group of genes. C-E. Hematoxylin and eosin staining of representative matured AO9-iPS-derived teratomas exhibiting characteristic structure of (C) intestinal epithelium (endoderm), (D) cartilage (mesoderm) and (E) neural epithelium (ectoderm).

FIG. 14 shows transgene silencing after AO9 reprogramming. Quantitative PCR for expression of retroviral transgenes in AO9-iPSC lines a, b and c, hADF, and hADF 6 days after the transduction with the two retroviruses (hADF-AO-6d). Mean values (n=3)±SEM are plotted indicating expression of the specific gene normalized to GAPDH/Actin relative to hADF expression, which was arbitrarily assigned a value of 0, in a logarithmic scale.

FIG. 15 shows the AO9-iPSC in vitro differentiation capacity. qRT-PCR data for differentiation markers GATA4 and AFP (endoderm), RUNX1 and BRACHURY (Mesoderm) and NCAM and NESTIN (ectoderm) at day 10 of in vitro differentiation protocol. Embryo bodies were derived from H9 hESCs or AO9 derived iPSCs. Average expression values±SEM are represented relative to undifferentiated H9-ESCs controls (normalized to GAPDH/Actin, logarithmic scale).

FIG. 16 shows neural lineage in vitro differentiation. A-D. Immunocytochemistry analysis of neuroprogenitor (NP) cell markers on cells derived after neural-differentiation protocol of EBs from AO9-iPSCs show the presence of NPs (A, C, D) as compared to the original hADF (B). A and B panels show double staining on the same slide. E. More differentiated neural cells were obtained from the previous NPs following specific GABAergic or dopaminergic differentiation protocols as shown in panels E. Specific markers for mature neuron (synapsin), GABAergic neuron (Calbindin) and dopaminergic neurons (Tyroxin Hydroxylase, TH) were used for immunoflurescence analysis.

FIG. 17 shows the role of ASF1A and OCT4 in H3K56 acetylation. A. Retroviral driven overexpression of ASF1A alone or ASF1A+OCT4 increases H3K56 acetylation in hADF shown by immunoprecipitation 72 hours after transduction using H3K56 antibodies (IP: H3Ac56) and gel blotted (Wb) with H3K56 antibody as well. H9-ESCs and AO9-iPSCs samples were used as positive control for Immunoprecipitation. B. Immunoprecipitation (IP) and western blot (Wb) using specific antibodies against H3K56ac (IP: H3Ac56) and ASF1A (Wb: ASF1A) demonstrate protein-protein interaction of ASF1A with acetylated H3K56 in transduced hADF. C. Protein interaction is observed between ASF1A and OCT4 when ASF1A is immunoprecipitated in hADF overexpressing OCT4+ASF1A; and in pluripotent cells H9-hESC; OSKM iPSCs and AO9-iPSC. Immunoprecipitated material was analyzed by western blot (Wb) using the specified antibodies to detect OCT4 and ASF1A coimmunoprecipitation. □-Actin was used as a loading control D. Chromatin immunoprecipitation assay in hADF overexpressing GFP, ASF1A, OCT4, both ASF1A and OCT4 and in H9 hESC and AO9-iPSCs using specific antibody against H3K56Ac. qRT-PCR was done using ChIP and input samples using the specific primers for NANOG, OCT4 and SOX2 promoters and two negative controls KRTHA4 (hypoacetylated gene) and an intergenic region primers. Mean values (n=3)±SEM are plotted indicating amplification of the specific gene region normalized to GFP sample, which was arbitrarily assigned a value of 1. Data correspond to the average of 3 independent experiments done in duplicate. T-student test was applied to determine statistical significance: ***P>0.001, **>0.05, *>0.01 compared to ASF1A expressing hADF.

FIG. 18 shows hADF transduced with retroviral vectors encoding OCT4 and ASF1A (bicistronic pMX retroviral vector co-expressing GFP and ASF1A) or (OCT4+ASF1A) or ASF1A and OCT4 alone, show different degrees of H3K56 acetylation being highest when ASF1 and OCT4 are used in combination followed by ASF1A alone, and OCT4 alone being the lowest. Immunocytochemistry using specific antibodies against H3K56ac, and OCT4 was used to analyze H3K56ac levels after GFP, GFP-ASF1A, OCT4 or both factors overexpression in hADF.

FIG. 19 shows the results of a chromatin immunoprecipitation assay in hADF overexpressing GFP, ASF1A, OCT4, both ASF1A and OCT4 and in H9 hESC and AO9-iPSCs using specific antibody against H3K56Ac. Specific region of NANOG, OCT4 and SOX2 promoter was amplified by PCR using the immunoprecipitated material to measure H3K56ac binding to these regions. Sample before immunoprecipitation was used as loading control (input) in the PCR.

FIG. 20 is a table showing the identification of significantly activated canonical pathways using Ingenuity Software (IKB) based on co-regulated genes between the four cell groups (AO9, ASF1A, OCT4 and GDF9).

FIG. 21 shows comparisons of differentially expressed genes 48 hours after overexpression of different factors in human dermal fibroblasts. Venn-diagrams to select the genes that are differentially expressed in AOG condition compared to OSKM (region II, upper diagram). Lower diagrams show three different comparison of previous region II with single factor specific up/downregulated genes.

FIG. 22 shows GDF9 signaling. A. hADF stimulated with GDF9 (500 ng/ul) or TGFb3 (20 ng/ul) at different times were lysed and western blot against phospho-smad2/3 (left panel) and total smad2/3 (right panel) was performed. B. Quantification of band pixel intensity showed a time dependent smad2/3 phosphorilation after GDF9 addition. C. Smad2/3 phosphorilation was no longer stimulated 48 hours after GDF9 addition as compared to 45 minutes. D. hADF stimulated with GDF9 (500 ng/ul) for 15 or 45 min were analyzed for p38-MapKinase phosphorilation by western blot.

FIG. 23 shows comparisons of differentially expressed genes 48 hours after overexpression of the identified factors in human dermal fibroblasts. Heatmap based on genes up- or down-regulated as compared to fibroblasts overexpressing GFP (hADF) or ASF1A only, OKSM only, ASF1+OCT4+GDF9 combined or exposure to GDF9 only.

FIG. 24 is a table showing gene ontology categories that are significantly represented in the genes regulated specifically by AO9 combination, by GDF9 treatment, by OCT4 overexpression or ASF1A overexpression.

### DETAILED DESCRIPTION

### General

In certain aspects, provided herein are methods and compositions for inducing a somatic cell to acquire a less differentiated phenotype and for generating induced pluripotent stem cells (iPS cells) by inducing expression of ASF1A in the cell and/or by contacting the cell with GDF9.

Notably, the methods described herein allow for the generation of pluripotent cell populations without the destruction of human embryos. Moreover, in certain embodiments, the methods described herein allow for the production of iPS cells without inducing expression of oncogenes c-MYC or KLF4. The cells created using the methods described herein are therefore particularly useful for use in regenerative medicine.

As described herein, ASF1A is a histone chaperone protein that is necessary for the cellular reprogramming of somatic cells into undifferentiated iPS cells. Notably, induced overexpression of ASF1A along with OCT3/4 in somatic cells results in the cells acquiring a more pluripotent phenotype and the production of iPS cells. ASF1A is therefore a newly discovered reprogramming factor that can be used in the generation of iPS cells.

Additionally, as described herein, contacting somatic cells to the oocyte-specific growth factor GDF9 enhances their reprogramming into pluripotent cells. For example, somatic cells in which expression of ASF1A and OCT3/4 is induced efficiently become iPS cells when cultured in the presence of GDF9 following induction of ASF1A and OCT3/4 expression. Thus, iPS cell generation can be improved by culturing cells undergoing dedifferentiation in the presence of GDF9.

In some aspects, provided herein are compositions and methods for treating and/or diagnosing cancer and for identifying agents useful in the treatment and/or diagnosis of cancer.

As described herein, inhibition of ASF1A causes pluripotent cells to acquire a more differentiated phenotype. Notably, acquisition of a more pluripotent phenotype is a hallmark of many forms of cancer. As such, inhibition of ASF1A is a useful method of treating or preventing cancer, while ASF1A inhibitors are useful as cancer therapeutics. Additionally, detection of ASF1A expression or activity can be used to determine whether an individual has cancer and/or is at high risk of acquiring cancer.

### Definitions

For convenience, certain terms employed in the specification, examples, and appended claims are collected here.

The articles “a” and “an” are used herein to refer to one or to more than one (i.e., to at least one) of the grammatical object of the article. By way of example, “an element” means one element or more than one element.

The term “agent” is used herein to denote a chemical compound, a small molecule, a mixture of chemical compounds and/or a biological macromolecule (such as a nucleic acid, an antibody, an antibody fragment, a protein or a peptide). Agents may be identified as having a particular activity by screening assays described herein below. The activity of such agents may render them suitable as a “therapeutic agent” which is a biologically, physiologically, or pharmacologically active substance (or substances) that acts locally or systemically in a subject.

As used herein, the term “cancer” includes, but is not limited to, solid tumors and blood borne tumors. The term cancer includes diseases of the skin, tissues, organs, bone, cartilage, blood and vessels. The term “cancer” further encompasses primary and metastatic cancers.

An “expression vector” is a vector which is capable of promoting expression of a nucleic acid incorporated therein. Typically, the nucleic acid to be expressed is “operably linked” to a transcriptional control element, such as a promoter and/or an enhancer, and is therefore subject to transcription regulatory control by the transcriptional control element.

As used herein, the terms “interfering nucleic acid,” “inhibiting nucleic acid” are used interchangeably. Interfering nucleic acids generally include a sequence of cyclic subunits, each bearing a base-pairing moiety, linked by intersubunit linkages that allow the base-pairing moieties to hybridize to a target sequence in a nucleic acid (typically an RNA) by Watson-Crick base pairing, to form a nucleic acid:oligomer heteroduplex within the target sequence. Interfering RNA molecules include, but are not limited to, antisense molecules, siRNA molecules, single-stranded siRNA molecules, miRNA molecules and shRNA molecules. Such an interfering nucleic acids can be designed to block or inhibit translation of mRNA or to inhibit natural pre-mRNA splice processing, or induce degradation of targeted mRNAs, and may be said to be “directed to” or “targeted against” a target sequence with which it hybridizes. Interfering nucleic acids may include, for example, peptide nucleic acids (PNAs), locked nucleic acids (LNAs), 2′-O-Methyl oligonucleotides and RNA interference agents (siRNA agents). RNAi molecules generally act by forming a herteroduplex with the target molecule, which is selectively degraded or “knocked down,” hence inactivating the target RNA. Under some conditions, an interfering RNA molecule can also inactivate a target transcript by repressing transcript translation and/or inhibiting transcription of the transcript. An interfering nucleic acid is more generally said to be “targeted against” a biologically relevant target, such as a protein, when it is targeted against the nucleic acid of the target in the manner described above.

The terms “polynucleotide”, and “nucleic acid” are used interchangeably. They refer to a polymeric form of nucleotides of any length, either deoxyribonucleotides or ribonucleotides, or analogs thereof. Polynucleotides may have any three-dimensional structure, and may perform any function. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. A polynucleotide may be further modified, such as by conjugation with a labeling component. In all nucleic acid sequences provided herein, U nucleotides are interchangeable with T nucleotides.

The phrase “pharmaceutically-acceptable carrier” as used herein means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body.

“Small molecule” as used herein, is meant to refer to a composition, which has a molecular weight of less than about 5 kD and most preferably less than about 4 kD. Small molecules can be nucleic acids, peptides, polypeptides, peptidomimetics, carbohydrates, lipids or other organic (carbon-containing) or inorganic molecules. Many pharmaceutical companies have extensive libraries of chemical and/or biological mixtures, often fungal, bacterial, or algal extracts, which can be screened with any of the assays described herein.

An oligonucleotide “specifically hybridizes” to a target polynucleotide if the oligomer hybridizes to the target under physiological conditions, with a Tm substantially greater than 45° C., or at least 50° C., or at least 60° C. to 80° C. or higher. Such hybridization corresponds to stringent hybridization conditions. At a given ionic strength and pH, the Tm is the temperature at which 50% of a target sequence hybridizes to a complementary polynucleotide. Again, such hybridization may occur with “near” or “substantial” complementarity of the antisense oligomer to the target sequence, as well as with exact complementarity.

As used herein, the term “subject” means a human or non-human animal selected for treatment or therapy.

The phrases “therapeutically-effective amount” and “effective amount” as used herein means the amount of an agent which is effective for producing the desired therapeutic effect in at least a sub-population of cells in a subject at a reasonable benefit/risk ratio applicable to any medical treatment.

“Treating” a disease in a subject or “treating” a subject having a disease refers to subjecting the subject to a pharmaceutical treatment, e.g., the administration of a drug, such that at least one symptom of the disease is decreased or prevented from worsening.

The term “vector” refers to the means by which a nucleic acid can be propagated and/or transferred between animals, cells, or cellular components. Vectors include plasmids, viruses, retroviruses, bacteriophage, pro-viruses, phagemids, transposons, and artificial chromosomes, and the like, that may or may not be able to replicate autonomously or integrate into a chromosome of a host cell. Vectors can be isolated, extracellular, extrachromosomal or can be integrated into the chromosomal DNA of a cell.

### ASF1A

In humans, ASF1A is encoded by the ASF1A gene. ASF1A is the most conserved member of the histone 3 and histone 4 chaperone proteins. ASF1A has been implicated in replication, transcription, and DNA repair. Most of the information about ASF1A comes from the work done in yeast and drosophila. It has been characterized as a histone-remodeling chaperone that cooperates with histone regulator A (HIRA) and with chromatin assembly factor 1 (CAF-1). ASF1A is required for H3K56 acetylation. An exemplary amino acid sequence of human ASF1A is provided at NCBI accession number NP_054753.1, which is hereby incorporated by reference. An exemplary nucleic acid sequence of human ASF1A mRNA is provided at NCBI accession number NM_014034.2, which is hereby incorporated by reference.

In certain embodiments, provided herein are methods and compositions for inducing a somatic cell to acquire a less differentiated phenotype and for generating iPS cells by inducing expression and/or activity of ASF1A. In some embodiments, expression of ASF1A is induced, for example, by transfecting the cell with an ASF1A expression vector, or by contacting the cell with an agent that causes the cell to express increased levels of ASF1A. In some embodiments, the cell is contacted directly with ASF1A protein.

Also provided herein are compositions and methods for treating cancer through the inhibition of ASF1A. For example. The expression and/or activity of ASF1A can be inhibited using small molecule ASF1A inhibitors or inhibitory nucleic acids that bind to ASF1A mRNA.

Also provided herein are methods of diagnosing cancer through the detection of ASF1A expression and/or activity. In some embodiments, ASF1A expression is detected, for example, by detecting ASF1A mRNA (e.g., using a ASF1A-specific nucleic acid probe and/or nucleic acid amplification reaction) and/or by detecting ASF1A protein (e.g., using an ASF1A specific antibody or antibody fragment). In some embodiments, ASF1A activity is detected, for example, by detecting H3K56 acetylation.

### GDF9

In certain embodiments, provided herein are methods and compositions for inducing a somatic cell to acquire a less differentiated phenotype and for generating iPS cells by contacting the cell with GDF9. In some embodiments, cells are contacted by GDF9 directly by adding GDF9 to cell culture media, or indirectly by inducing GDF9 expression by the cell. In some embodiments, GDF9 expression is induced in feeder cells that are included in culture with the cells that are being dedifferentiated.

GDF9 (Growth/Differentiation Factor 9) is encoded by the GDF9 gene. GDF9 is a member of the TGFβ superfamily that is expressed in oocytes and plays a role in ovarian folliculogenesis. GDF9 is expressed as several different isoform variants. Exemplary amino acid sequences of the isoform variants of human GDF9 are provided at NCBI accession numbers NP 001275753.1, NP_001275754.1, NP_001275755.1, NP_001275756.1, NP_001275757.1 and NP_005251.1, each of which is hereby incorporated by reference. Exemplary nucleic acid sequences of the isoform variants of human GDF9 mRNA are provided at NCBI accession numbers NM 01288824.2, NM 01288825.2, NM 01288826.2, NM 01288827.2, NM_01288828.2 and NM_005260.4, each of which is hereby incorporated by reference.

### Other Reprogramming Factors

In certain embodiments of the methods provided herein, expression of one or more reprogramming factors is induced in a cell. In some embodiments, reprogramming factor expression is induced, for example, by transfecting the cell with an expression vector encoding the reprogramming factor, or by contacting the cell with an agent that causes the cell to express increased levels of the reprogramming factor. In some embodiments, the cell is contacted with reprogramming factor protein directly. Examples of reprogramming factors used in the methods described herein include, but are not limited to, OCT3/4, NANOG, SOX1, SOX2, SOX3, SOX15, SOX18, DNMT3B, c-MYC, N-MYC, L-MYC, KLF1, KLF2, KLF4, KLF5, LIN28 and/or GLIS1.

OCT3/4 is also known as POU class 5 homobox 1 (POU5F1). Exemplary amino acid sequences of isoform variants of human OCT3/4 are provided at NCBI accession numbers NP_00167002.1, NP_001272915.1, NP_001272916.1, NP_002692.2 and NP_976034.4, each of which is hereby incorporated by reference. Exemplary nucleic acid sequences of isoform variants of human OCT3/4 mRNA are provided at NCBI accession numbers NM_001173531.2, NM_001285986.1, NM_001285987.1, NM_002701.5 and NM_203289.5, each of which is hereby incorporated by reference.

An exemplary amino acid sequence NANOG is provided at NCBI accession number XP_005253541.1, which is hereby incorporated by reference. An exemplary nucleic acid sequences of human NANOG mRNA is provided at NCBI accession number XM-005253484.2, which is hereby incorporated by reference.

SOX1 is also known as sex determining region Y-box 1(SRY-box 1). An exemplary amino acid sequence SOX1 is provided at NCBI accession number NP_005977.2, which is hereby incorporated by reference. An exemplary nucleic acid sequences of human SOX1 mRNA is provided at NCBI accession number NM_005986.2, which is hereby incorporated by reference.

SOX2 is also known as sex determining region Y-box 2 (SRY-box 2). An exemplary amino acid sequence SOX2 is provided at NCBI accession number NP_003097.1, which is hereby incorporated by reference. An exemplary nucleic acid sequences of human SOX2 mRNA is provided at NCBI accession number NM_003106.3, which is hereby incorporated by reference.

SOX3 is also known as sex determining region Y-box 3 (SRY-box 3). An exemplary amino acid sequence SOX3 is provided at NCBI accession number NP_005625.2, which is hereby incorporated by reference. An exemplary nucleic acid sequences of human SOX3 mRNA is provided at NCBI accession number NM_005634.2, which is hereby incorporated by reference.

SOX15 is also known as sex determining region Y-box 15 (SRY-box 15). An exemplary amino acid sequence SOX15 is provided at NCBI accession number NP_008873.1, which is hereby incorporated by reference. An exemplary nucleic acid sequences of human SOX15 mRNA is provided at NCBI accession number NM_006942.1, which is hereby incorporated by reference.

SOX18 is also known as sex determining region Y-box 18 (SRY-box 18). An exemplary amino acid sequence SOX18 is provided at NCBI accession number NP_060889.1, which is hereby incorporated by reference. An exemplary nucleic acid sequences of human SOX18 mRNA is provided at NCBI accession number NM_018419.2, which is hereby incorporated by reference.

Exemplary amino acid sequences of isoform variants of human DNMT3B are provided at NCBI accession numbers NP_001193984.1, NP_001193985.1, NP_008823.1, NP_787044.1, NP_787045.1 and NP_787046.1, each of which is hereby incorporated by reference. Exemplary nucleic acid sequences of isoform variants of human DNMT3B mRNA are provided at NCBI accession numbers NM_001207055.1, NM_001207056.1, NM_006892.3, NM_175848.1, NM_175849.1 and NM_175850.2, each of which is hereby incorporated by reference.

c-MYC is also known as v-myc avian myelocytomatosis viral oncogene homolog (MYC). An exemplary amino acid sequence c-MYC is provided at NCBI accession number NP_002458.2, which is hereby incorporated by reference. An exemplary nucleic acid sequences of human c-MYC mRNA is provided at NCBI accession number NM_002467.4, which is hereby incorporated by reference.

N-MYC is also known as v-myc avian myelocytomatosis viral oncogene neuroblastoma derived homolog (MYCN). Exemplary amino acid sequences of isoform variants of human N-MYC are provided at NCBI accession numbers NP_001280157.1, NP_001280160.1, NP_001280162.1 and NP_005369.2, each of which is hereby incorporated by reference. Exemplary nucleic acid sequences of isoform variants of human N-MYC mRNA are provided at NCBI accession numbers NM_001293228.1, NM_001293231.1, NM_001293233.1 and NM_005378.5, each of which is hereby incorporated by reference.

L-MYC is also known as v-myc avian myelocytomatosis viral oncogene lung carcinoma derived homolog (MYCL). Exemplary amino acid sequences of isoform variants of human L-MYC are provided at NCBI accession numbers NP_001028253.1, NP_001028254.2 and NP_005367.2, each of which is hereby incorporated by reference. Exemplary nucleic acid sequences of isoform variants of human L-MYC mRNA are provided at NCBI accession numbers NM_001033081.2, NM_001033082.2 and NM_005376.4, each of which is hereby incorporated by reference.

An exemplary amino acid sequence KLF1 is provided at NCBI accession number NP_006554.1, which is hereby incorporated by reference. An exemplary nucleic acid sequences of human KLF1 mRNA is provided at NCBI accession number NM_006563.3, which is hereby incorporated by reference.

An exemplary amino acid sequence KLF2 is provided at NCBI accession number NP_016270.2, which is hereby incorporated by reference. An exemplary nucleic acid sequences of human KLF2 mRNA is provided at NCBI accession number NM_057354.1, which is hereby incorporated by reference.

An exemplary amino acid sequence KLF4 is provided at NCBI accession number NP_004226.3, which is hereby incorporated by reference. An exemplary nucleic acid sequences of human KLF4 mRNA is provided at NCBI accession number NM_004235.4, which is hereby incorporated by reference.

Exemplary amino acid sequences of isoform variants of human KLF5 are provided at NCBI accession numbers NP_00127347.1 and NP_001721.2, each of which is hereby incorporated by reference. Exemplary nucleic acid sequences of isoform variants of human KLF5 mRNA are provided at NCBI accession numbers NM_001286818.1 and NM_001730.4, each of which is hereby incorporated by reference.

LIN28 is also known lin-28 homolog A (LIN28A). Exemplary amino acid sequences of isoform variants of human LIN28 are provided at NCBI accession numbers XP_006710963.1 and XP_006710962.1, each of which is hereby incorporated by reference. Exemplary nucleic acid sequences of isoform variants of human LIN28 mRNA are provided at NCBI accession numbers XM_006710900.1 and XM_006710899.1, each of which is hereby incorporated by reference.

An exemplary amino acid sequence GLIS1 is provided at NCBI accession number NP_671726.2, which is hereby incorporated by reference. An exemplary nucleic acid sequences of human GLIS1 mRNA is provided at NCBI accession number NM_147193.2, which is hereby incorporated by reference.

### Generation of Induced Pluripotent Stem Cells

In certain aspects, provided herein are methods (e.g., in vitro methods) of inducing a somatic cell to acquire a less differentiated phenotype (e.g., for the generation of iPS cells). In some embodiments, the method includes the step of inducing expression of ASF1A in a somatic cell. In some embodiments, the cell is contacted with GDF9.

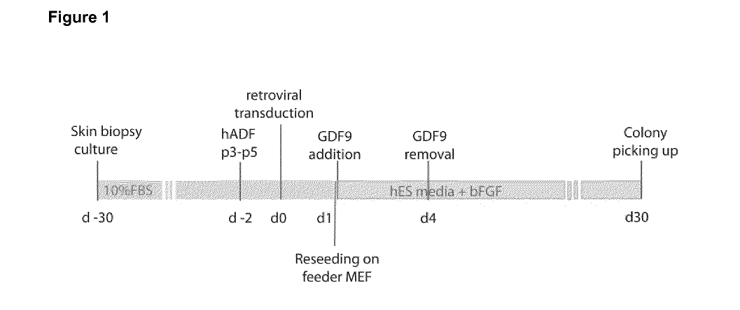

A schematic depiction of an exemplary method of producing iPS cells from somatic cells is depicted in FIG. 1. According to this exemplary method, Low passage a human adult dermal fibroblasts (hADFs) are seeded (e.g., at 100,000 cells/well) and infected with vectors (e.g., retroviral vectors) encoding OCT3/4 (e.g., pMX-OCT4) and ASF1A (e.g., pMX-ASF1A). In some embodiments, the cells are infected in the presence of polybrene (e.g., 4 μg/ml polybrene). In some embodiments, after about 24 hours, cells are re-plated (e.g., onto six-well plates) on a feeder layer of feeder cells (e.g., mitomycin C-treated mouse embryonic fibroblasts). In some embodiments, at this point the culture medium is changed to hES medium (e.g., DMEM/F12 containing 20% KSR, 10 ng/ml of human recombinant basic fibroblast growth factor (bFGF), 1×NEAA, 1×L-Glutamine, 5.5 mM 2-ME, penicillin and streptomycin) containing GDF9 (e.g., 100, 200, 300, 400, 500, 600, 700, 800, 900 or 1000 nM). In some embodiments, cells are cultured in GDF9 containing media for at least 12, 24, 36, 48, 60, 72, 84 or 96 hours, after which cells are cultured in hES medium. Colonies of iPS cells may appear 14-21 days after transduction. In some embodiments, the iPS cell lines are confirmed as being positive for Tra-1-60, SSEA-4 and/or NANOG by immunofluorescence.

In some embodiment, any somatic cell can be used in the methods disclosed herein. In some embodiments the cell is a vertebrate cell, such as a mammalian cell including non-primate cells (e.g., cells from a cow, pig, horse, donkey, goat, camel, cat, dog, guinea pig, rat, mouse, sheep) and primate cells (e.g., a cell from a monkey, gorilla, chimpanzee). In some embodiments, the cell is a human cell. In some embodiments the cell is a primary cell. In some embodiments, the cell is a fibroblast, an osteoblast, a chondroblast, a myoblast, a lipoblast, an interstitial cell, an angioblast, a juxtaglomerular cell, a stromal cell, a sertoli cell, a lymphocyte, a myeloid cell, an endothelial progenitor cell, a trichocyte, a gonadotrope, a neuron, a chromaffin cell, a melanocyte, an odontoblast, a corneal keratocyte, an ependymocyte or a pinealocyte. In some embodiments, the cell is a human adult dermal fibroblast (hADF). In some embodiments, the cell is from a cell line (e.g., P19 cells, HUVAC cells, 293-T cells, 3T3 cells, 721 cells, 9L cells, A2780 cells, A172 cells, A253 cells, A431 cells, CHO cells, COS-7 cells, HCA2 cells, HeLa cells, Jurkat cells, NIH-3T3 cells and Vero cells).

In some embodiments the method includes inducing expression of one or more reprogramming factors in the cell (e.g., OCT3/4, NANOG, SOX1, SOX2, SOX3, SOX15, SOX18, DNMT3B, c-MYC, N-MYC, L-MYC, KLF1, KLF2, KLF4, KLF5, LIN28 and/or GLIS1). In some embodiments, expression of ASF1A and OCT3/4 is induced in the cell. In some embodiments, ASF1A, OCT3/4, SOX2, KLF4 and cMYC are expressed in the cell. In some embodiments, the expression of ASF1A and/or one or more other reprogramming factors is induced in the cell by contacting the cell with one or more expression vectors encoding the reprogramming factor(s). In some embodiments the expression vector is a retroviral vector (e.g., a self-inactivating retroviral vector). In some embodiments the expression vector is a lentiviral vector. In some embodiments, the expression vector is an adenovirus vector. In some embodiments, the expression vector is a plasmid vector. In some embodiments, the expression vector is linear DNA.

In some embodiments, reprogramming factor protein compounds are introduced into the cell in combination with and/or instead of an expression vector. In some embodiments, expression of ASF1A and/or one or more reprogramming factors is induced by contacting the cell with an agent that induces expression of the reprogramming factor. In some embodiments, the cell is contacted with one or more agents that enhance the dedifferentiation process (e.g., a histone deacetylase inhibitor, such as valproic acid, a histone methyl transferase inhibitor, such as BIX-01294, an ALK5 inhibitor such as SB431412, a MEK inhibitor such as PD0325901).

In some embodiments, the cell is contacted with GDF9 after expression of one or more reprogramming factors is induced in the cell (e.g., about 1 day after induction of expression of the reprogramming factor(s)). In some embodiments, the cell is contacted with GDF9 (e.g., 100, 200, 300, 400, 500, 600, 700, 800, 900 or 1000 nM GDF9) about 1, 2, 3, 4, 5, 6 or 7 days after induction of expression of the reprogramming factor(s). In some embodiments, the cells are contacted with GDF9 for at least 12, 24, 36, 48, 60, 72, 84 or 96 hours.

In some embodiments the method includes culturing the cell under conditions whereby the cell acquires a less differentiated phenotype (e.g., becomes an iPS cell). In some embodiments the cell is cultured in human ES cell medium (e.g., DMEM/F12 containing 20% KSR, 10 ng/ml of human recombinant basic fibroblast growth factor (bFGF), 1×NEAA, 1×L-Glutamine, 5.5 mM 2-ME, penicillin and streptomycin). In some embodiments, the human ES cell medium includes GDF9 for at least a portion of the time the cell is being cultured. In some embodiments, the cell is cultured with feeder cells (e.g., mitomycin-C treated mouse fibroblasts). In some embodiments, the feeder cells express GDF9.

### ASF1A Inhibitors

Certain embodiments described herein relate to methods of treating and/or preventing cancer. These methods involve administering an agent that inhibits ASF1A. For example, such agents may inhibit the activity and/or expression of ASF1A. Agents which may be used to inhibit the ASF1A pathway and/or ASF1A include proteins, peptides, small molecules and inhibitory RNA molecules, e.g., siRNA molecules, shRNA, ribozymes, and antisense oligonucleotides.

Any agent that inhibits ASF1A and/or the ASF1A pathway can be used to practice certain methods described herein. Such agents can be those described herein, those known in the art, or those identified through screening assays (e.g. the screening assays described herein).

In some embodiments, assays used to identify agents useful in the methods described herein include a reaction between ASF1A and one or more assay components. The other components may be, for example, a test agent (e.g. the potential agent), or a combination of a test agent and a ASF1A target (e.g. histone H3 and/or histone H4). Agents identified via such assays, such as those described herein, may be useful, for example, for treating and/or preventing cancer.

Agents useful in the methods described herein may be obtained from any available source, including systematic libraries of natural and/or synthetic compounds. Agents may also be obtained by any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive; see, e.g., Zuckermann et al., 1994, J. Med. Chem. 37:2678-85); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam, 1997, Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of agents may be presented in solution (e.g., Houghten, 1992, Biotechniques 13:412-421), or on beads (Lam, 1991, Nature 354:82-84), chips (Fodor, 1993, Nature 364:555-556), bacteria and/or spores, (Ladner, U.S. Pat. No. 5,223,409), plasmids (Cull et al, 1992, Proc Natl Acad Sci USA 89:1865-1869) or on phage (Scott and Smith, 1990, Science 249:386-390; Devlin, 1990, Science 249:404-406; Cwirla et al, 1990, Proc. Natl. Acad. Sci. 87:6378-6382; Felici, 1991, J. Mol. Biol. 222:301-310; Ladner, supra.).

Agents useful in the methods described herein may be identified, for example, using assays for screening candidate or test compounds which inhibit the formation of a conjugate between ASF1A or a biologically active portion thereof and a ASF1A target.

In some embodiments, the assay systems used to identify compounds that modulate the activity of ASF1A involves preparing a reaction mixture containing ASF1A and a ASF1A target under conditions and for a time sufficient to allow ASF1A to conjugate to its substrate. For example, such conditions can be established through the use of a concentrated cell extract. Use of such extracts are described, for example, in the exemplification and in Merbl and Kirschner, Proc Natl Acat Sci USA 106:2543-2548 (2009), which is hereby incorporated by reference in its entirety. In some embodiments a tissue sample, such as a tumor sample, is used to establish conditions to facilitate conjugation of ASF1A to its target. In some embodiments, the ASF1A and/or the ASF1A target is linked, either directly or indirectly, to a detectable moiety (e.g., a radioactive, fluorescent, luminescent and/or enzymatic moiety) to facilitate its detection. In order to test an agent for activity, a reaction mixture is prepared in the presence of the compound and a control reaction mixture is prepared in the absence of the test compound. The control reaction mixture may also contain a placebo agent. The test compound can be initially included in the reaction mixture, or can be added at a time subsequent to the addition of ASF1A and its target. Control reaction mixtures are incubated without the test compound or with a placebo. The conjugation of the substrate by ASF1A is then detected. Target conjugation can be detected by any method known in the art including, but not limited to, using anti-ASF1A antibodies and/or detectably labeled ASF1A and/or target to detect the level of conjugation. Conjugation of the target in the control reaction, but less or no such conjugation in the reaction mixture containing the test compound, indicates that the compound decreases with the activity of ASF1A.

The assay for agents that inhibit the interaction of ASF1A with its binding partner may be conducted in a heterogeneous or homogeneous format. Heterogeneous assays involve anchoring either ASF1A or its target onto a solid phase and detecting conjugates anchored to the solid phase at the end of the reaction. In homogeneous assays, the entire reaction is carried out in a liquid phase. In either approach, the order of addition of reactants can be varied to obtain different information about the agents being tested. For example, test compounds that interfere with the interaction between ASF1A and the binding partner (e.g., by competition) can be identified by conducting the reaction in the presence of the test substance, i.e., by adding the test substance to the reaction mixture prior to or simultaneously with ASF1A and its interactive binding partner. Alternatively, test compounds that disrupt preformed conjugates can be tested by adding the test compound to the reaction mixture after conjugates have been formed. The various formats are briefly described below.

In a heterogeneous assay system, either ASF1A or its target is anchored onto a solid surface or matrix, while the other corresponding non-anchored component may be labeled, either directly or indirectly. In practice, microtitre plates are often utilized for this approach. The anchored species can be immobilized by a number of methods, either non-covalent or covalent, that are well known in the art. Non-covalent attachment can often be accomplished simply by coating the solid surface with a solution of ASF1A or its target and drying. Alternatively, an immobilized antibody specific for the assay component to be anchored can be used for this purpose.

A homogeneous assay may also be used to identify inhibitors of ASF1A. This is typically a reaction, analogous to those mentioned above, which is conducted in a liquid phase in the presence or absence of the test agent. The formed conjugates are then separated from unconjugated components, and the amount of conjugate formed is determined. As mentioned for heterogeneous assay systems, the order of addition of reactants to the liquid phase can yield information about which test compounds inhibit conjugate formation and which disrupt preformed conjugates.

In such a homogeneous assay, the reaction products may be separated from unreacted assay components by any of a number of standard techniques, including but not limited to: differential centrifugation, chromatography, electrophoresis and immunoprecipitation. In differential centrifugation, conjugates of molecules may be separated from unconjugated molecules through a series of centrifugal steps, due to the different sedimentation equilibria of conjugates based on their different sizes and densities (see, for example, Rivas, G., and Minton, A. P., Trends Biochem Sci 1993 August; 18(8):284-7). Standard chromatographic techniques may also be utilized to separate conjugated molecules from unconjugated ones. For example, gel filtration chromatography separates molecules based on size, and through the utilization of an appropriate gel filtration resin in a column format, for example, the relatively larger conjugate may be separated from the relatively smaller unconjugated components. Immunoprecipitation is another common technique utilized for the isolation of a protein-protein conjugates from solution (see, e.g., Ausubel et al (eds.), In: Current Protocols in Molecular Biology, J. Wiley & Sons, New York. 1999). In this technique, all proteins binding to an antibody specific to one of the binding molecules are precipitated from solution by conjugating the antibody to a bead that may be readily collected by centrifugation or through the application of a magnetic field. The bound assay components may be released from the beads, and a second immunoprecipitation step performed, this time utilizing antibodies specific for the correspondingly different interacting assay component. Alternatively, the presence of the second assay component in the immunoprecipitated fraction can detected directly using a detectable label, for example, a detectable label linked either directly or indirectly to ASF1A or its target.

In another embodiment, agents useful in the methods described herein may be identified using assays for screening candidate or test compounds which bind to ASF1A or a biologically active portion thereof. Determining the ability of the test agent to directly bind to ASF1A can be accomplished, for example, by coupling the compound with a detectable label such that binding of the compound to ASF1A can be determined by detecting the labeled compound in a complex. For example, compounds can be labeled with ¹²⁵I, ³⁵S, ¹⁴C, or ³H, either directly or indirectly, and the radioisotope detected by direct counting of radioemission or by scintillation counting. Alternatively, assay components can be enzymatically labeled with, for example, horseradish peroxidase, alkaline phosphatase, or luciferase, and the enzymatic label detected by determination of conversion of an appropriate substrate to product.

Modulators of ASF1A expression may also be identified, for example, using methods wherein a cell is contacted with a candidate compound and the expression of ASF1A mRNA or protein is determined. The level of expression of mRNA or protein in the presence of the candidate compound is compared to the level of expression of mRNA or protein in the absence of the candidate compound. The candidate compound can then be identified as a modulator of ASF1A expression based on this comparison. For example, when expression of ASF1A is greater in the presence of the candidate compound than in its absence, the candidate compound is identified as a stimulator of ASF1A mRNA or protein expression. Conversely, when expression of ASF1A is less in the presence of the candidate compound than in its absence, the candidate compound is identified as an inhibitor of ASF1A mRNA or protein expression.

### Interfering Nucleic Acids

In certain embodiments, interfering (i.e., inhibiting) nucleic acid molecules that selectively target ASF1A are provided herein and/or used in methods described herein. Interfering nucleic acids generally include a sequence of cyclic subunits, each bearing a base-pairing moiety, linked by intersubunit linkages that allow the base-pairing moieties to hybridize to a target sequence in a nucleic acid (typically an RNA) by Watson-Crick base pairing, to form a nucleic acid:oligomer heteroduplex within the target sequence. Interfering RNA molecules include, but are not limited to, antisense molecules, siRNA molecules, single-stranded siRNA molecules, miRNA molecules and shRNA molecules.

Typically at least 17, 18, 19, 20, 21, 22 or 23 nucleotides of the complement of the target mRNA sequence are sufficient to mediate inhibition of a target transcript. Perfect complementarity is not necessary. In some embodiments, the interfering nucleic acid molecule is double-stranded RNA. The double-stranded RNA molecule may have a 2 nucleotide 3′ overhang. In some embodiments, the two RNA strands are connected via a hairpin structure, forming a shRNA molecule. shRNA molecules can contain hairpins derived from microRNA molecules. For example, an RNAi vector can be constructed by cloning the interfering RNA sequence into a pCAG-miR30 construct containing the hairpin from the miR30 miRNA. RNA interference molecules may include DNA residues, as well as RNA residues. In some embodiments, the interfering nucleic acid is a single-stranded antisense nucleic acid (e.g., RNA).

Interfering nucleic acid molecules provided herein can contain RNA bases, non-RNA bases or a mixture of RNA bases and non-RNA bases. For example, interfering nucleic acid molecules provided herein can be primarily composed of RNA bases but also contain DNA bases or non-naturally occurring nucleotides.

The interfering nucleic acids can employ a variety of oligonucleotide chemistries. Examples of oligonucleotide chemistries include, without limitation, peptide nucleic acid (PNA), linked nucleic acid (LNA), phosphorothioate, 2′O-Me-modified oligonucleotides, and morpholino chemistries, including combinations of any of the foregoing. In general, PNA and LNA chemistries can utilize shorter targeting sequences because of their relatively high target binding strength relative to 2′O-Me oligonucleotides. Phosphorothioate and 2′O-Me-modified chemistries are often combined to generate 2′O-Me-modified oligonucleotides having a phosphorothioate backbone. See, e.g., PCT Publication Nos. WO/2013/112053 and WO/2009/008725, incorporated by reference in their entireties.

Peptide nucleic acids (PNAs) are analogs of DNA in which the backbone is structurally homomorphous with a deoxyribose backbone, consisting of N-(2-aminoethyl) glycine units to which pyrimidine or purine bases are attached. PNAs containing natural pyrimidine and purine bases hybridize to complementary oligonucleotides obeying Watson-Crick base-pairing rules, and mimic DNA in terms of base pair recognition (Egholm, Buchardt et al. 1993). The backbone of PNAs is formed by peptide bonds rather than phosphodiester bonds, making them well-suited for antisense applications (see structure below). The backbone is uncharged, resulting in PNA/DNA or PNA/RNA duplexes that exhibit greater than normal thermal stability. PNAs are not recognized by nucleases or proteases.

Despite a radical structural change to the natural structure, PNAs are capable of sequence-specific binding in a helix form to DNA or RNA. Characteristics of PNAs include a high binding affinity to complementary DNA or RNA, a destabilizing effect caused by single-base mismatch, resistance to nucleases and proteases, hybridization with DNA or RNA independent of salt concentration and triplex formation with homopurine DNA. PANAGENE® has developed its proprietary Bts PNA monomers (Bts; benzothiazole-2-sulfonyl group) and proprietary oligomerization process. The PNA oligomerization using Bts PNA monomers is composed of repetitive cycles of deprotection, coupling and capping. PNAs can be produced synthetically using any technique known in the art. See, e.g., U.S. Pat. Nos. 6,969,766, 7,211,668, 7,022,851, 7,125,994, 7,145,006 and 7,179,896. See also U.S. Pat. Nos. 5,539,082; 5,714,331; and 5,719,262 for the preparation of PNAs. Further teaching of PNA compounds can be found in Nielsen et al., Science, 254:1497-1500, 1991. Each of the foregoing is incorporated by reference in its entirety.

Interfering nucleic acids may also contain “locked nucleic acid” subunits (LNAs). “LNAs” are a member of a class of modifications called bridged nucleic acid (BNA). BNA is characterized by a covalent linkage that locks the conformation of the ribose ring in a C30-endo (northern) sugar pucker. For LNA, the bridge is composed of a methylene between the 2′-O and the 4′-C positions. LNA enhances backbone preorganization and base stacking to increase hybridization and thermal stability.

The structures of LNAs can be found, for example, in Wengel, et al., Chemical Communications (1998) 455; Tetrahedron (1998) 54:3607, and Accounts of Chem. Research (1999) 32:301); Obika, et al., Tetrahedron Letters (1997) 38:8735; (1998) 39:5401, and Bioorganic Medicinal Chemistry (2008) 16:9230. Compounds provided herein may incorporate one or more LNAs; in some cases, the compounds may be entirely composed of LNAs. Methods for the synthesis of individual LNA nucleoside subunits and their incorporation into oligonucleotides are described, for example, in U.S. Pat. Nos. 7,572,582, 7,569,575, 7,084,125, 7,060,809, 7,053,207, 7,034,133, 6,794,499, and 6,670,461, each of which is incorporated by reference in its entirety. Typical intersubunit linkers include phosphodiester and phosphorothioate moieties; alternatively, non-phosphorous containing linkers may be employed. One embodiment is an LNA containing compound where each LNA subunit is separated by a DNA subunit. Certain compounds are composed of alternating LNA and DNA subunits where the intersubunit linker is phosphorothioate.

“Phosphorothioates” (or S-oligos) are a variant of normal DNA in which one of the nonbridging oxygens is replaced by a sulfur. The sulfurization of the internucleotide bond reduces the action of endo- and exonucleases including 5′ to 3′ and 3′ to 5′ DNA POL 1 exonuclease, nucleases S1 and P1, RNases, serum nucleases and snake venom phosphodiesterase. Phosphorothioates are made by two principal routes: by the action of a solution of elemental sulfur in carbon disulfide on a hydrogen phosphonate, or by the method of sulfurizing phosphite triesters with either tetraethylthiuram disulfide (TETD) or 3H-1, 2-bensodithiol-3-one 1, 1-dioxide (BDTD) (see, e.g., Iyer et al., J. Org. Chem. 55, 4693-4699, 1990). The latter methods avoid the problem of elemental sulfur's insolubility in most organic solvents and the toxicity of carbon disulfide. The TETD and BDTD methods also yield higher purity phosphorothioates.

“2′O-Me oligonucleotides” molecules carry a methyl group at the 2′-OH residue of the ribose molecule. 2′-O-Me-RNAs show the same (or similar) behavior as DNA, but are protected against nuclease degradation. 2′-O-Me-RNAs can also be combined with phosphothioate oligonucleotides (PTOs) for further stabilization. 2′O-Me oligonucleotides (phosphodiester or phosphothioate) can be synthesized according to routine techniques in the art (see, e.g., Yoo et al., Nucleic Acids Res. 32:2008-16, 2004).

The interfering nucleic acids described herein may be contacted with a cell or administered to an organism (e.g., a human). Alternatively, constructs and/or vectors encoding the interfering RNA molecules may be contacted with or introduced into a cell or organism. In certain embodiments, a viral, retroviral or lentiviral vector is used. In some embodiments, the vector has a tropism for cardiac tissue. In some embodiments the vector is an adeno-associated virus.

Typically at least 17, 18, 19, 20, 21, 22 or 23 nucleotides of the complement of the target mRNA sequence are sufficient to mediate inhibition of a target transcript. Perfect complementarity is not necessary. In some embodiments, the interfering nucleic acids contains a 1, 2 or 3 nucleotide mismatch with the target sequence. The interfering nucleic acid molecule may have a 2 nucleotide 3′ overhang. If the interfering nucleic acid molecule is expressed in a cell from a construct, for example from a hairpin molecule or from an inverted repeat of the desired sequence, then the endogenous cellular machinery will create the overhangs. shRNA molecules can contain hairpins derived from microRNA molecules. For example, an RNAi vector can be constructed by cloning the interfering RNA sequence into a pCAG-miR30 construct containing the hairpin from the miR30 miRNA. RNA interference molecules may include DNA residues, as well as RNA residues.

In some embodiments, the interfering nucleic acid molecule is a siRNA molecule. Such siRNA molecules should include a region of sufficient homology to the target region, and be of sufficient length in terms of nucleotides, such that the siRNA molecule down-regulate target RNA. The term “ribonucleotide” or “nucleotide” can, in the case of a modified RNA or nucleotide surrogate, also refer to a modified nucleotide, or surrogate replacement moiety at one or more positions. It is not necessary that there be perfect complementarity between the siRNA molecule and the target, but the correspondence must be sufficient to enable the siRNA molecule to direct sequence-specific silencing, such as by RNAi cleavage of the target RNA. In some embodiments, the sense strand need only be sufficiently complementary with the antisense strand to maintain the overall double-strand character of the molecule.

In addition, an siRNA molecule may be modified or include nucleoside surrogates. Single stranded regions of an siRNA molecule may be modified or include nucleoside surrogates, e.g., the unpaired region or regions of a hairpin structure, e.g., a region which links two complementary regions, can have modifications or nucleoside surrogates. Modification to stabilize one or more 3′- or 5′-terminus of an siRNA molecule, e.g., against exonucleases, or to favor the antisense siRNA agent to enter into RISC are also useful. Modifications can include C3 (or C6, C7, C12) amino linkers, thiol linkers, carboxyl linkers, non-nucleotidic spacers (C3, C6, C9, C12, abasic, triethylene glycol, hexaethylene glycol), special biotin or fluorescein reagents that come as phosphoramidites and that have another DMT-protected hydroxyl group, allowing multiple couplings during RNA synthesis.

Each strand of an siRNA molecule can be equal to or less than 35, 30, 25, 24, 23, 22, 21, or 20 nucleotides in length. In some embodiments, the strand is at least 19 nucleotides in length. For example, each strand can be between 21 and 25 nucleotides in length. In some embodiments, siRNA agents have a duplex region of 17, 18, 19, 29, 21, 22, 23, 24, or 25 nucleotide pairs, and one or more overhangs, such as one or two 3′ overhangs, of 2-3 nucleotides.

A “small hairpin RNA” or “short hairpin RNA” or “shRNA” includes a short RNA sequence that makes a tight hairpin turn that can be used to silence gene expression via RNA interference. The shRNAs provided herein may be chemically synthesized or transcribed from a transcriptional cassette in a DNA plasmid. The shRNA hairpin structure is cleaved by the cellular machinery into siRNA, which is then bound to the RNA-induced silencing complex (RISC).

In some embodiments, shRNAs are about 15-60, 15-50, or 15-40 (duplex) nucleotides in length, about 15-30, 15-25, or 19-25 (duplex) nucleotides in length, or are about 20-24, 21-22, or 21-23 (duplex) nucleotides in length (e.g., each complementary sequence of the double-stranded shRNA is 15-60, 15-50, 15-40, 15-30, 15-25, or 19-25 nucleotides in length, or about 20-24, 21-22, or 21-23 nucleotides in length, and the double-stranded shRNA is about 15-60, 15-50, 15-40, 15-30, 15-25, or 19-25 base pairs in length, or about 18-22, 19-20, or 19-21 base pairs in length). shRNA duplexes may comprise 3′ overhangs of about 1 to about 4 nucleotides or about 2 to about 3 nucleotides on the antisense strand and/or 5′-phosphate termini on the sense strand. In some embodiments, the shRNA comprises a sense strand and/or antisense strand sequence of from about 15 to about 60 nucleotides in length (e.g., about 15-60, 15-55, 15-50, 15-45, 15-40, 15-35, 15-30, or 15-25 nucleotides in length), or from about 19 to about 40 nucleotides in length (e.g., about 19-40, 19-35, 19-30, or 19-25 nucleotides in length), or from about 19 to about 23 nucleotides in length (e.g., 19, 20, 21, 22, or 23 nucleotides in length).

Non-limiting examples of shRNA include a double-stranded polynucleotide molecule assembled from a single-stranded molecule, where the sense and antisense regions are linked by a nucleic acid-based or non-nucleic acid-based linker; and a double-stranded polynucleotide molecule with a hairpin secondary structure having self-complementary sense and antisense regions. In some embodiments, the sense and antisense strands of the shRNA are linked by a loop structure comprising from about 1 to about 25 nucleotides, from about 2 to about 20 nucleotides, from about 4 to about 15 nucleotides, from about 5 to about 12 nucleotides, or 1, 2, 3, 4, 5, 6, 7, 8, 9, 10, 11, 12, 13, 14, 15, 16, 17, 18, 19, 20, 21, 22, 23, 24, 25, or more nucleotides.

Additional embodiments related to the shRNAs, as well as methods of designing and synthesizing such shRNAs, are described in U.S. patent application publication number 2011/0071208, the disclosure of which is herein incorporated by reference in its entirety for all purposes.

In some embodiments, provided herein are micro RNAs (miRNAs). miRNAs represent a large group of small RNAs produced naturally in organisms, some of which regulate the expression of target genes. miRNAs are formed from an approximately 70 nucleotide single-stranded hairpin precursor transcript by Dicer. miRNAs are not translated into proteins, but instead bind to specific messenger RNAs, thereby blocking translation. In some instances, miRNAs base-pair imprecisely with their targets to inhibit translation.

In some embodiments, antisense oligonucleotide compounds are provided herein. In certain embodiments, the degree of complementarity between the target sequence and antisense targeting sequence is sufficient to form a stable duplex. The region of complementarity of the antisense oligonucleotides with the target RNA sequence may be as short as 8-11 bases, but can be 12-15 bases or more, e.g., 10-40 bases, 12-30 bases, 12-25 bases, 15-25 bases, 12-20 bases, or 15-20 bases, including all integers in between these ranges. An antisense oligonucleotide of about 14-15 bases is generally long enough to have a unique complementary sequence.

In certain embodiments, antisense oligonucleotides may be 100% complementary to the target sequence, or may include mismatches, e.g., to improve selective targeting of allele containing the disease-associated mutation, as long as a heteroduplex formed between the oligonucleotide and target sequence is sufficiently stable to withstand the action of cellular nucleases and other modes of degradation which may occur in vivo. Hence, certain oligonucleotides may have about or at least about 70% sequence complementarity, e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence complementarity, between the oligonucleotide and the target sequence. Oligonucleotide backbones that are less susceptible to cleavage by nucleases are discussed herein. Mismatches, if present, are typically less destabilizing toward the end regions of the hybrid duplex than in the middle. The number of mismatches allowed will depend on the length of the oligonucleotide, the percentage of G:C base pairs in the duplex, and the position of the mismatch(es) in the duplex, according to well understood principles of duplex stability.

Interfering nucleic acid molecules can be prepared, for example, by chemical synthesis, in vitro transcription, or digestion of long dsRNA by Rnase III or Dicer. These can be introduced into cells by transfection, electroporation, or other methods known in the art. See Hannon, G J, 2002, RNA Interference, Nature 418: 244-251; Bernstein E et al., 2002, The rest is silence. RNA 7: 1509-1521; Hutvagner G et al., RNAi: Nature abhors a double-strand. Curr. Opin. Genetics & Development 12: 225-232; Brummelkamp, 2002, A system for stable expression of short interfering RNAs in mammalian cells. Science 296: 550-553; Lee N S, Dohjima T, Bauer G, Li H, Li M-J, Ehsani A, Salvaterra P, and Rossi J. (2002). Expression of small interfering RNAs targeted against HIV-1 rev transcripts in human cells. Nature Biotechnol. 20:500-505; Miyagishi M, and Taira K. (2002). U6-promoter-driven siRNAs with four uridine 3′ overhangs efficiently suppress targeted gene expression in mammalian cells. Nature Biotechnol. 20:497-500; Paddison P J, Caudy A A, Bernstein E, Hannon G J, and Conklin D S. (2002). Short hairpin RNAs (shRNAs) induce sequence-specific silencing in mammalian cells. Genes & Dev. 16:948-958; Paul C P, Good P D, Winer I, and Engelke D R. (2002). Effective expression of small interfering RNA in human cells. Nature Biotechnol. 20:505-508; Sui G, Soohoo C, Affar E-B, Gay F, Shi Y, Forrester W C, and Shi Y. (2002). A DNA vector-based RNAi technology to suppress gene expression in mammalian cells. Proc. Natl. Acad. Sci. USA 99(6):5515-5520; Yu J-Y, DeRuiter S L, and Turner D L. (2002). RNA interference by expression of short-interfering RNAs and hairpin RNAs in mammalian cells. Proc. Natl. Acad. Sci. USA 99(9):6047-6052.

In the present methods, an interfering nucleic acid molecule or an interfering nucleic acid encoding polynucleotide can be administered to the subject, for example, as naked nucleic acid, in combination with a delivery reagent, and/or as a nucleic acid comprising sequences that express an interfering nucleic acid molecule. In some embodiments the nucleic acid comprising sequences that express the interfering nucleic acid molecules are delivered within vectors, e.g. plasmid, viral and bacterial vectors. Any nucleic acid delivery method known in the art can be used in the methods described herein. Suitable delivery reagents include, but are not limited to, e.g., the Mirus Transit TKO lipophilic reagent; lipofectin; lipofectamine; cellfectin; polycations (e.g., polylysine), atelocollagen, nanoplexes and liposomes. The use of atelocollagen as a delivery vehicle for nucleic acid molecules is described in Minakuchi et al. Nucleic Acids Res., 32(13):e109 (2004); Hanai et al. Ann NY Acad Sci., 1082:9-17 (2006); and Kawata et al. Mol Cancer Ther., 7(9):2904-12 (2008); each of which is incorporated herein in their entirety. Exemplary interfering nucleic acid delivery systems are provided in U.S. Pat. Nos. 8,283,461, 8,313,772, 8,501,930, 8,426,554, 8,268,798 and 8,324,366, each of which is hereby incorporated by reference in its entirety.

In some embodiments of the methods described herein, liposomes are used to deliver an inhibitory oligonucleotide to a subject. Liposomes suitable for use in the methods described herein can be formed from standard vesicle-forming lipids, which generally include neutral or negatively charged phospholipids and a sterol, such as cholesterol. The selection of lipids is generally guided by consideration of factors such as the desired liposome size and half-life of the liposomes in the blood stream. A variety of methods are known for preparing liposomes, for example, as described in Szoka et al. (1980), Ann. Rev. Biophys. Bioeng. 9:467; and U.S. Pat. Nos. 4,235,871, 4,501,728, 4,837,028, and 5,019,369, the entire disclosures of which are herein incorporated by reference.

The liposomes for use in the present methods can also be modified so as to avoid clearance by the mononuclear macrophage system (“MMS”) and reticuloendothelial system (“RES”). Such modified liposomes have opsonization-inhibition moieties on the surface or incorporated into the liposome structure.

Opsonization-inhibiting moieties for use in preparing the liposomes described herein are typically large hydrophilic polymers that are bound to the liposome membrane. As used herein, an opsonization inhibiting moiety is “bound” to a liposome membrane when it is chemically or physically attached to the membrane, e.g., by the intercalation of a lipid-soluble anchor into the membrane itself, or by binding directly to active groups of membrane lipids. These opsonization-inhibiting hydrophilic polymers form a protective surface layer that significantly decreases the uptake of the liposomes by the MMS and RES; e.g., as described in U.S. Pat. No. 4,920,016, the entire disclosure of which is herein incorporated by reference.

In some embodiments, opsonization inhibiting moieties suitable for modifying liposomes are water-soluble polymers with a number-average molecular weight from about 500 to about 40,000 daltons, or from about 2,000 to about 20,000 daltons. Such polymers include polyethylene glycol (PEG) or polypropylene glycol (PPG) derivatives; e.g., methoxy PEG or PPG, and PEG or PPG stearate; synthetic polymers such as polyacrylamide or poly N-vinyl pyrrolidone; linear, branched, or dendrimeric polyamidoamines; polyacrylic acids; polyalcohols, e.g., polyvinylalcohol and polyxylitol to which carboxylic or amino groups are chemically linked, as well as gangliosides, such as ganglioside GM1. Copolymers of PEG, methoxy PEG, or methoxy PPG, or derivatives thereof, are also suitable. In addition, the opsonization inhibiting polymer can be a block copolymer of PEG and either a polyamino acid, polysaccharide, polyamidoamine, polyethyleneamine, or polynucleotide. The opsonization inhibiting polymers can also be natural polysaccharides containing amino acids or carboxylic acids, e.g., galacturonic acid, glucuronic acid, mannuronic acid, hyaluronic acid, pectic acid, neuraminic acid, alginic acid, carrageenan; aminated polysaccharides or oligosaccharides (linear or branched); or carboxylated polysaccharides or oligosaccharides, e.g., reacted with derivatives of carbonic acids with resultant linking of carboxylic groups. In some embodiments, the opsonization-inhibiting moiety is a PEG, PPG, or derivatives thereof. Liposomes modified with PEG or PEG-derivatives are sometimes called “PEGylated liposomes.”

### Pharmaceutical Compositions

In certain embodiments, provided herein is a composition, e.g., a pharmaceutical composition, containing at least one agent described herein together with a pharmaceutically acceptable carrier. In one embodiment, the composition includes a combination of multiple (e.g., two or more) agents described herein.

As described in detail below, the pharmaceutical compositions disclosed herein may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; or (2) parenteral administration, for example, by subcutaneous, intramuscular, intravenous or epidural injection as, for example, a sterile solution or suspension, or sustained-release formulation.

Methods of preparing these formulations or compositions include the step of bringing into association an agent described herein with the carrier and, optionally, one or more accessory ingredients. In general, the formulations are prepared by uniformly and intimately bringing into association an agent described herein with liquid carriers, or finely divided solid carriers, or both, and then, if necessary, shaping the product.

Pharmaceutical compositions suitable for parenteral administration comprise one or more agents described herein in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

Regardless of the route of administration selected, the agents provided herein, which may be used in a suitable hydrated form, and/or the pharmaceutical compositions disclosed herein, are formulated into pharmaceutically-acceptable dosage forms by conventional methods known to those of skill in the art.

### Therapeutic Methods

Provided herein are methods of treatment of diseases and disorders that can be improved by disrupting the expression or activity of ASF1A. In some embodiments, described herein are therapeutic methods of treating cancer, including a cancerous tumor, comprising administering to a subject, (e.g., a subject in need thereof), an effective amount of an agent that inhibits ASF1A.

The pharmaceutical compositions described herein can be delivered by any suitable route of administration, including orally, nasally, as by, for example, a spray, rectally, intravaginally, parenterally, intracisternally and topically, as by powders, ointments or drops, including buccally and sublingually. In certain embodiments the pharmaceutical compositions are delivered generally (e.g., via oral or parenteral administration). In certain other embodiments the pharmaceutical compositions are delivered locally through direct injection into a tumor by direct injection into the tumor's blood supply (e.g., arterial or venous blood supply).

In certain embodiments, the methods of treatment described herein include administering an agent that inhibits ASF1A in conjunction with a second therapeutic agent to the subject. For example, when used for treating cancer, such methods may comprise administering pharmaceutical compositions described herein in conjunction with one or more chemotherapeutic agents and/or scavenger compounds, including chemotherapeutic agents described herein, as well as other agents known in the art. When used to treat immune disorders, such methods may include administering pharmaceutical compositions described herein in conjunction with one or more agents useful for the treatment of immune disorders, such as immunosuppressants or other therapeutic agents known in the art.

Conjunctive therapy includes sequential, simultaneous and separate, or co-administration of the active compound in a way that the therapeutic effects of the first agent administered have not entirely disappeared when the subsequent agent is administered. In certain embodiments, the second agent may be co-formulated with the first agent or be formulated in a separate pharmaceutical composition.

In some embodiments, the subject pharmaceutical compositions described herein will incorporate the substance or substances to be delivered in an amount sufficient to deliver to a patient a therapeutically effective amount of an incorporated therapeutic agent or other material as part of a prophylactic or therapeutic treatment. The desired concentration of the active compound in the particle will depend on absorption, inactivation, and excretion rates of the drug as well as the delivery rate of the compound. It is to be noted that dosage values may also vary with the severity of the condition to be alleviated. It is to be further understood that for any particular subject, specific dosage regimens should be adjusted over time according to the individual need and the professional judgment of the person administering or supervising the administration of the compositions. Typically, dosing will be determined using techniques known to one skilled in the art.

In certain embodiments, described herein are therapeutic methods of treating cancer in a subject in need thereof. A subject in need thereof may include, for example, a subject who has been diagnosed with a tumor, including a pre-cancerous tumor, a cancer, or a subject who has been treated, including subjects that have been refractory to the previous treatment.

The methods described herein may be used to treat any cancerous or pre-cancerous tumor. In certain embodiments, the tumor has increased expression of ASF1A protein or mRNA relative to non-tumor tissue (e.g., a non-tumor tissue of the same tissue type as the tumor). Cancers that may treated, prevented or diagnosed by methods and compositions described herein include, but are not limited to, cancer cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangio sarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

### Exemplification

### Materials and Methods

### Antibodies

The following antibodies were used in the experiments described herein: goat anti-OCT4 (IF assays), mouse anti-OCT4 (IP assays), rabbit anti-NANOG, rabbit LIN-28 from Transduction Laboratories (Lexington, Ky.), mouse phospho-p38 MAPKp (Thr180/Tyr182), phosphoSMAD2/3 (ser465/467), rabbit anti-ASF1A and Histone3-lysine56-Acetylated from Cell Signaling Technology (Beverly, Mass.), rabbit anti-SOX2, rabbit anti Calbindin D28K, mouse anti-TRA-1-60 from Chemicon/Millipore, anti-tyrosine hydroxylase (Pel-Freez), rabbit anti-DDX4 (Abcam), mouse anti-b-ACTIN (sigma), rabbit anti PAX-6 (Covance), rabbit anti-SSEA1 and anti-SSEA4 from Developmental Studies Hybridoma bank (Iowa).

### Vectors

The cDNAs encoding hASF1A (Open Biosystems) were subcloned into the self-inactivating retroviral bicistronic vector pMX-GFP (Cell Biolabs, INC). Lentiviral pWPI-ASF1A was made by PmeI restriction of pWPI vector (http://www.addgene.org/12254/) and human ASF1A cDNA was inserted. Lentiviral vector pLenti-shRNA-GFP encoding shRNA for ASF1A was purchased from Applied Biological Materials Inc. The shRNA-147 target sequence was: AAGTGAAGAATACGATCAAGT. The shRNA-1234 target sequence was: GGTCACAAGATTCCACATTAATTGGGAAG. The shRNA-4 target sequence was GCAAAGGTTCAGGTGAACAATGTAGTGGT. The shRNA-238 target sequence was AATCCAGGACTCATTCCAGAT. DNA vectors pMX-GFP, pMX-OCT4, pMX-SOX2, pMX-KLF4 and pMX-cMYC (H. sapiens) were purchased from Addgene.

### Cell Culture

H9 human ES cells (Wicell) and iPS cells were cultured in standard human ES cell culture medium (DMEM/F12 containing 20% KSR, 10 ng/ml of human recombinant basic fibroblast growth factor (bFGF), 1×NEAA, 1×L-Glutamine, 5.5 mM 2-ME, penicillin and streptomycin. ES cells and iPS cells were cultured on top of mitomycin-C mouse fibroblasts and picked mechanically.

Derivation of Human Adult Dermal Fibroblasts (hADF)

Primary skin fibroblasts were obtained via a 4-mm full-thickness skin punch biopsy from the upper back of healthy volunteers following informed consent. Cultured outgrowths appeared after 7-14 days. hADF were culture in DMEM containing 10% FBS, 1×NEAA, 1×L-Glutamine, penicillin and streptomycin.

### Production of Viral Supernatants

Hek293T cells were plated at 90% cell confluence in a 10-cm dish. The next day, cells were transfected with 10 μg viral vector, 7 μg Gag-Pol vector and 3 μg VSV-G plasmid using the polyethylenimine method. Supernatant was collected 24 h and 48 h post-transfection and filtered through 45-mm pore size filters. Viral titers were determined using Hek293T cells. Five ml of viral supernatant was used to infect 25,000 cells in the presence of 4 μg/ml polybrene.

### Reprogramming Assays

Low passage hADFs were seeded at 100,000 cells/well and infected with retroviral supernatants encoding OSKM factors (pMX-OCT4, pMX-Sox2, pMX-KLF4 and pMX-cMYC), or OSKM plus ASF1A (pMX-ASF1A) in the presence of 4 μg/ml polybrene. After 24 hours, cells were re-plated onto six-well plates on a feeder layer of mitomycin C-treated mouse embryonic fibroblasts (Millipore). Medium (hES medium) was changed daily. Colonies appeared 14-21 days after transduction. The iPS lines were confirmed positive for Tra-1-60, SSEA-4 and NANOG by immunofluorescence. In all fully reprogrammed iPSCs vector-encoded transgenes were found to be silenced.

For AO9 iPSC generation, the same protocol was followed but only OCT4 and ASF1A retroviral supernatants were added to hADF (FIG. 1). After 24 hours, cells were re-plated onto six-well plates on a feeder layer of mitomycin C-treated mouse embryonic fibroblasts (Millipore) and media was changed to hES medium in the presence of GDF9 500 nM (Sigma) and were kept into this media for 48 hours after transduction. Medium was changed daily.

### In Vitro Differentiation

Pluripotent cells differentiation was induced by culturing ES cells as EBs in low attachment plates with hES media in the absence of bFGF for 7 days. EBs were transferred to 0.1% gelatin-coated dishes and cultured in differentiation medium (KO DMEM supplemented with 10% fetal bovine serum, 1×MEM nonessential amino acids, 2 mML-glutamine, and 50 uM-mercaptoethanol) for up to 7 days. For the generation of neurons, neural progenitors were induced to differentiate by changing neural proliferation medium to neural differentiation medium (without bFGF) supplemented with 250 ng/ml SHH (R&D Systems), 100 ng/ml DKK1 (R&D Systems), 20 ng/ml BDNF (Peprotech) and 10 μM Y27632 (Calbiochem). After 19 days in the condition above, cells were exposed to 0.5 mM dibutryl-cyclic AMP (Sigma), 0.5 μM valpromide (Alfa Aesar), 20 ng/ml BDNF, 10 μM all-trans retinoic acid (RA, from Sigma) and 10 μM Y27632 for an additional 3 days.

### Proliferation Assay

Proliferation assays were performed using the CyQuant kit (Molecular Probes) according to manufacturer's instructions. For these assays, hADFs were cultured in DMEM 10% FBS. Cells were plated at a density of 20,000 cells per 24 well plate, and cell numbers were measured using a microplate reader. Experiments were performed in triplicate.

qRT-PCR Assay

RNA was isolated using a RNeasy kit (Qiagen) according to the manufacturer's instructions. First-strand cDNA was primed using oligo-dT oligonucleotides and RT-PCR was performed using the primer sets described herein. For quantitative RT-PCR, brilliant SYBR green was used for detection (Biorad).

### Chromatin Immunoprecipitation (ChIP)

For performance of ChIP assays, hADF cells were transduced with the identified factor and changed to hES medium. After 72 hours, 2 million cells were washed twice with PBS and collected following incubation in trypsin (0.25%). Protein was cross-linked to DNA by treatment with formaldehyde 0.5% for 15 min at room temperature, after which the reaction was stopped with 150 mM glycine. Pellets were resuspended in ChIP lysis buffer. Cells were sonicated using a Branson Sonifier 450D (Branson, Danbury, Conn., http://www.sonifer.com) at 50% amplitude, with 6 1-minute pulses in ice water. Samples were pre-cleared using protein G Dynabeads (Dynal Biotech, Carlsbad, Calif., http://www.invitrogen.com/dynal) in 1 ml of dilution buffer. Cell extracts were incubated overnight at 4° C. with 5 μL of H3K56Acetylated anti-serum (Cell Signaling) or 2 μg of rabbit non-specific IgG (Millipore). Chromatin antibody complexes were isolated using 50 μL of protein G Dynabeads and washed one time with low-salt buffer, one time with high-salt buffer, one time with LiCl wash buffer, and twice with TE buffer. Protein/DNA complexes were eluted from the beads at 65° C. with occasional vortexing. Crosslinking was reversed by addition of NaCl and incubation overnight at 65° C. Extracts were then treated with RNase A and proteinase K, and DNA was purified using an Upstate EZ ChIP kit (Millipore). PCR was performed on ChIP DNA and Input DNA. The following primer pair was used to analyze the Oct4 promoter region: forward 5′-TGAACTGTGGTGGAGAGTGC-3′ and reverse 5′-AGGAAGGGCTAGGACGAGAG-3′. Negative control primers for an intergenic region were the following: forward 5′-TTTTCAGTTCACACATATAAAGCAGA-3′ and reverse 5′-TGTTGTTGTTGTTGCTTCACTG-3′.

### Co-Immunoprecipitation and Western Blot Assay

Two million hADFs transduced with the different factors were used for each immunoprecipitation assay 72 hours after transduction. For ASF1A-OCT4 co-immunoprecipitation, cells were resuspended and incubated for 30 minutes RT with 5 mM DTBP. The pellet was resuspended in quenching buffer (100 mM Tris pH 8.0, 150 mM NaCl) and washed twice with PBS before cell lysis with 1% igepal (NP-40). For H3K56Ac immunoprecipitation, cells were resuspended for 15 minutes in paraformaldehyde and quenched with 2.5 M glycine. Cells were lysed in the presence of 20 mM sodium butirate and 1% igepal (NP-40). For immunoprecipitation, 1 mg of cell lysate (200 μl) was diluted to 500 μl in lysis buffer (50 mM HEPES, 150 mM NaCl, 1 mM EDTA, 2.5 mM EGTA, 10% glycerol, 1% Igepal) containing protease inhibitors and incubated overnight with 5 ul of rabbit anti-serum (OCT4 or H3K56Ac) or an equivalent amount of rabbit IgG at 4° C. Following overnight incubation, stable complexes were affinity purified by incubation with 50 ml of Protein-G Fast Flow agarose beads (Millipore) for 4 hours at 4° C. Beads bound to immunoprecipitated complexes were washed once in lysis buffer and twice in PBS. Bound proteins were eluted from the beads by boiling in 2× Laemmli buffer and size fractionated using SDS-PAGE. Desired protein was detected by Western blot analysis using an affinity purified antibody. For smad2/3 and MAP kinase western blots, cells were washed with ice-cold PBS and lysed in 1% Igepal buffer (50 mM HEPES pH 7.4, 10 mM EDTA, 150 mM NaCl, 10 mM sodium pyrophosphate, 100 mM sodium fluoride, 1 mM sodium vanadate and a tablet of complete protease inhibitor cocktail(Roche). After centrifugation at 12,000×g for 15 min, 100 ug of protein supernatant was resuspended in Laemmli SDS-DTT sample buffer for western blot analysis using the identified antibodies.

### Microarray Analysis

Global gene expression profiles were obtained using the Illumina HumanHT-12 v4.0 Expression BeadChip (San Diego, Calif.) covering well-characterized genes, gene candidates, and splice variants with over 47,000 probes. Data normalization and differential gene expression analyses were done using Illumina's GenomeStudio Gene Expression Module requiring a fold-change of at least 2.0 and significant detection (p<0.05) for the gene in the sample the gene is up-regulated.

Gene Ontology (GO) analysis was done using Expression Analysis Systematic Explorer (EASE), which identified biologically relevant categories that were over-represented in the input gene set. EASE identifies GO categories in the input gene list that are over represented using jackknife iterative resampling of Fisher exact probabilities, with Bonferroni multiple testing correction. The “EASE score” is the upper bound of the distribution of Jackknife Fisher exact probabilities, which is a significance level with smaller EASE scores indicating increasing confidence in overrepresentation. We picked GO categories that have EASE scores of 0.05 or lower as significantly over-represented.

Pathway analysis was done using Ingenuity Software Knowledge Base (IKB), (Redwood City, Calif.) to identify pathways that are significantly activated for a given input gene list. The association p-value between an input gene list and a known pathway is calculated using right-tailed Fisher Exact Test. We picked pathways that had a p-value of less than 0.05 after Benjamini-Hochberg correction for multiple hypothesis testing.

### Primers

The following primer pairs were used herein:

| | Forward | Reverse |
|---|---|---|
| ASF1A cloning | ggcgcttgTTTAAACCCggCACCATgGCA | GGCCGAAGGGTTTAAAccctcaCATGCAG |
| | AAGGTTCAGGTGAA | TCCATGTGGG |
| POU5F1 endog | CCTCACTTCACTGCACTGTA | CAGGTTTTCTTTCCCTAGCT |
| POU5F1 transgene | CCCCAGGGCCCCATTTTGGTACC | CTTCCCTCCAACCAGTTGCCCCAAAC |
| POU5F1 | GGTTCTATTTGGGAAGGTAT | CATGTTCTTGAAGCTAAGC |
| ASF1A transgene | TTTAAACCCggCACCatggca | GCATCTGCATCTGGAATGAG |
| ASF1A | CCGCAGGAAGGCATATGTT | GCATCTGCATCTGGAATGAG |
| actin | TGAAGTGTGACGTGGACATC | GGAGGAGCAATGATCTTGAT |
| hTERT | TGTGCACCAACATCTACAAG | GCGTTCTTGGCTTTCAGGAT |
| hGDF3 | AAATGTTTGTGTTGCGGTCA | TCTGGCACAGGTGTCTTCAG |
| SOX2 | CCCAGCAGACTTCACATGT | CCTCCCATTTCCCTCGTTTT |
| KLF4 | GATGAACTGACCAGGCACTA | GTGGGTCATATCCACTGTCT |
| DNMT3B | ATAAGTCGAAGGTGCGTCGT | GGCAACATCTGAAGCCATTT |
| NANOG | TACCTCAGCCTCCAGCAGAT | TCTGGAACCAGGTCTTCACC |
| GAPDH | ATGGAAATCCCATCACCATCTT | CGG CCC ACT TGA TTT TGG |
| REX1 | CCCACAGTCCATCCTTACAGAGTT | GGG ACT TTG CCC CCA AAC |
| RUNX1 | CCCTAGGGGATGTTCCAGAT | TGAAGCTTTTCCCTCTTCCA |
| BRACHURY | ACCACCGCTGGAAATATGTGAACG | AACTCTCACGATGTGAATCCGAGG |
| NESTIN | CAGCGTTGGAACAGAGGTTGG | TGGCACAGGTGTCTCAAGGGTAG |
| AFP | AGCTTGGTGGTGGATGAAAC | CCCTCTTCAGCAAAGCAGAC |
| NCAM | ATGGAAACTCTATTAAAGTGAACCTG | TAGACCTCATACTCAGCATTCCAGT |
| PAX6 | CGGAGTGAATCAGCTCGGTG | CCGCTTATACTGGGCTATTTTGC |
| GATA4 | CTCTACATGAAGCTCCAC | CTGCTGGTGTCTTAGATT |
| ChIP NANOG F | GAAAGACATGACAAATCACCAGAC | CAACTAGCTCCATTTTCCTCTTTC |
| ChIP SOX2 | CGGTTGAATGAAGACAGTCTAGTG | CGACTAGAAGTTAGGAGACCCAAA |
| ChIP OCt4 | TTACTTAAGTCGACAGAGGTCAGC | TGGTCTAGTGCTTGATTCTGTTTG |
| ChIP KRTHA4 | TAGGTATACTCCCATCCATTCCAT | TAGCAGAAACTCAACCTGTATTCG |
| ChIP Intergenic | AATGAGTGGGCTCATGGAAA | TCTGGATGCAGCATTTGTGT |

### Example 1: ASF1A is Expressed in Pluripotent Cell Populations

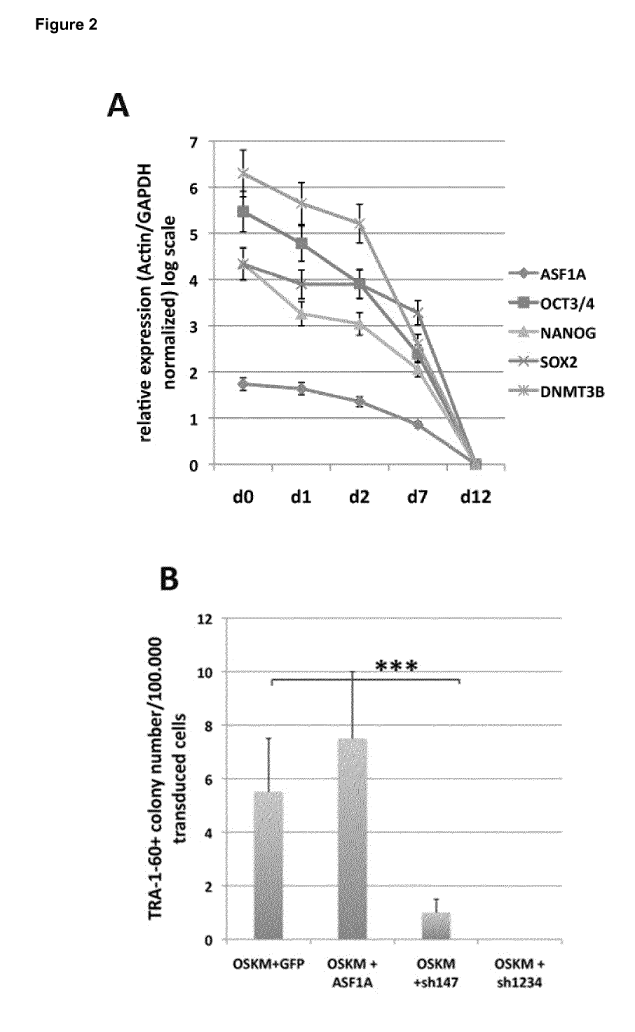

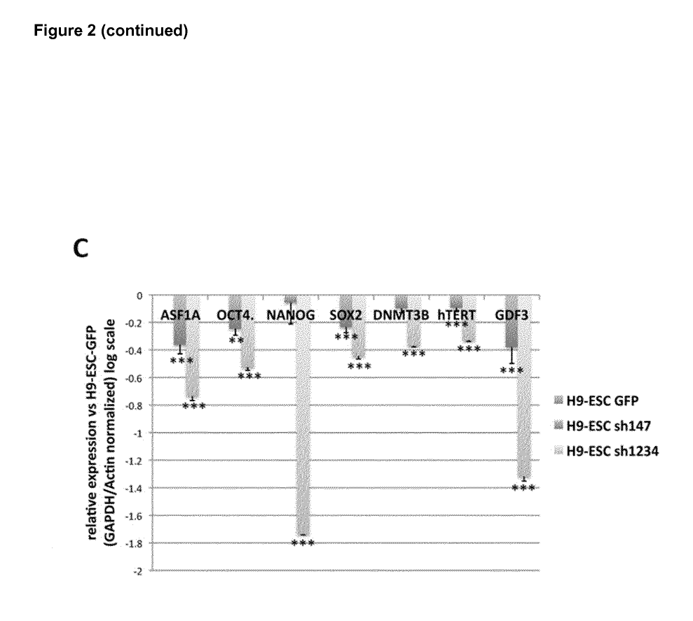

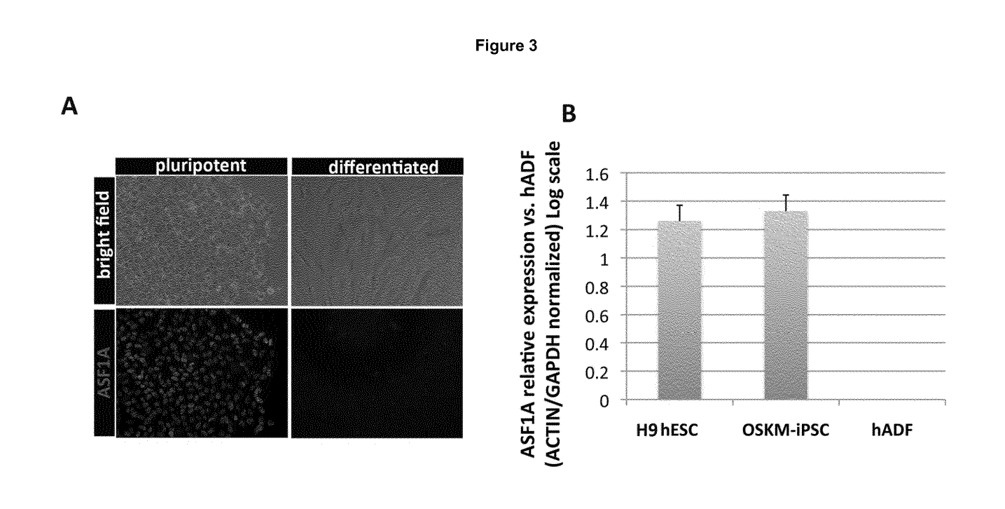

To determine if ASF1A has a role in acquisition of pluripotency, its expression in hESCs during differentiation was examined. Gene expression and protein analyses revealed that during spontaneous differentiation ASF1A expression decreased, as did the expression of pluripotency-related genes OCT3/4, NANOG, SOX2, and DNMT3B (FIGS. 2A and 3A). The highest ASF1A expression levels were observed in hESCs and the lowest in hADFs (FIG. 3B).

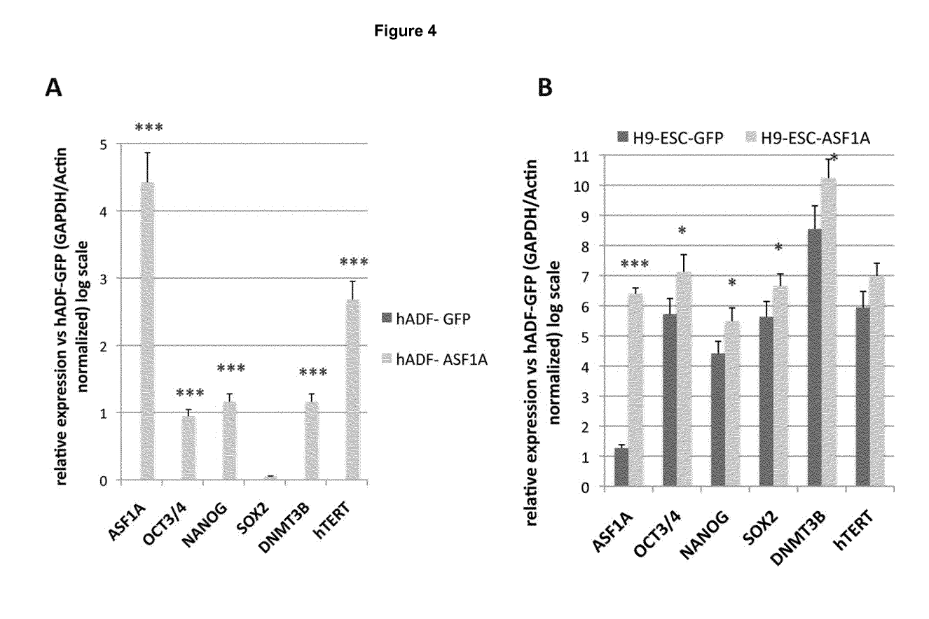

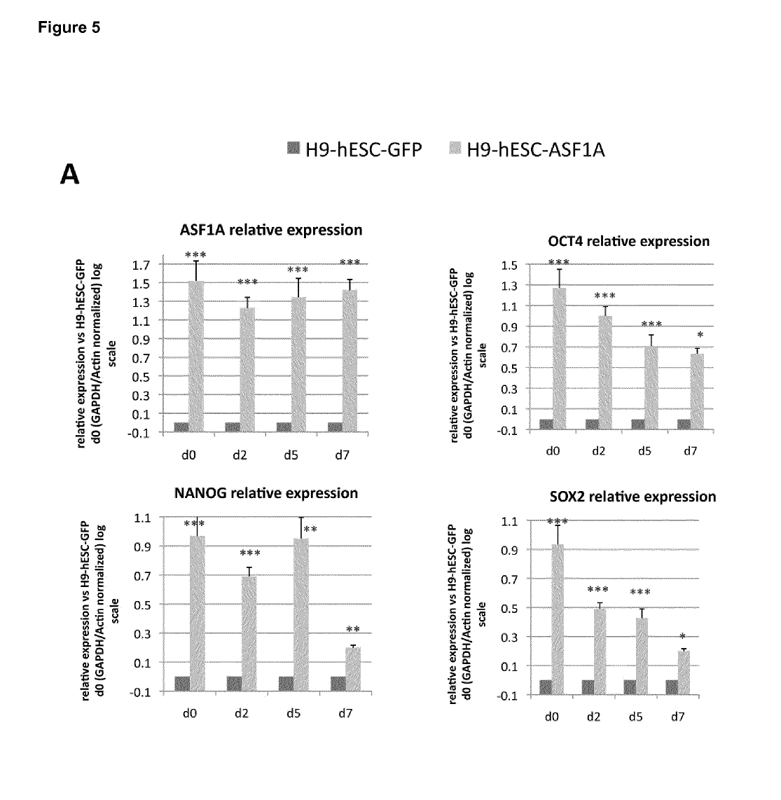

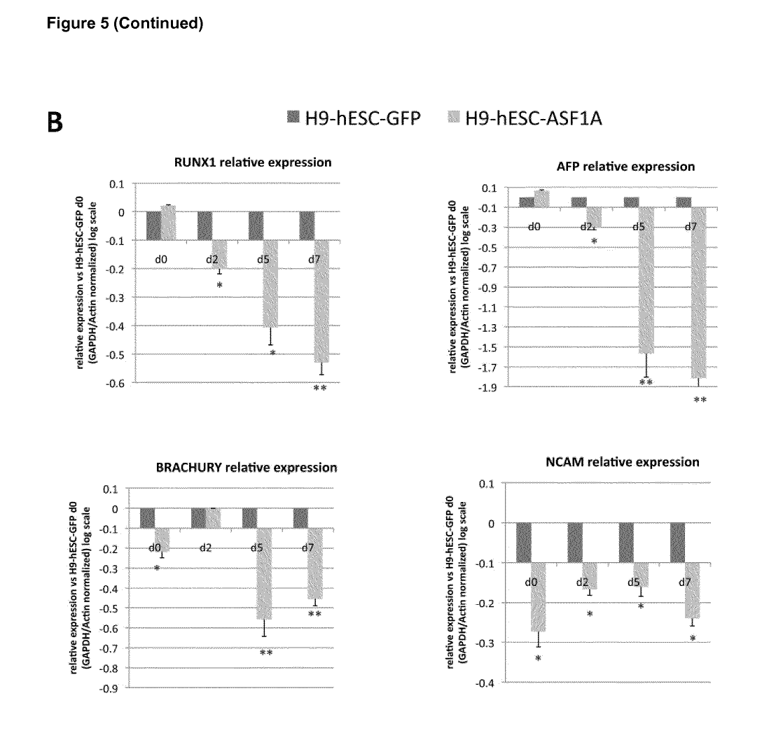

To further investigate the role of ASF1A in somatic and embryonic stem cells, whether forced expression of ASF1A in hESCs and hADFs would affect their differentiated states was examined. H9 hESCs and hADF were engineered to over-express either ASF1A or GFP by transducing these cells with a lentiviral vector (pWPI). H9 hESCs overexpressing ASF1A showed a tenfold increase in the expression of pluripotency marker genes OCT4, NANOG, SOX2, and DNMT3B (FIG. 4B) 6 days after transduction. hADFs overexpressing ASF1A also showed a similar relative increase in pluripotency marker expression compared to GFP transduced cells (FIG. 4A). When hESCs overexpressing ASF1A were cultured as embryo bodies and then plated in 10% FBS media to promote spontaneous differentiation into endoderm, mesoderm, and ectoderm cell derivatives, ASF1A-overexpressing hESCs showed a clear resistance to differentiation by delaying the downregulation of pluripotency-related genes and the onset of expression of differentiation markers (FIGS. 5A and 5B). These results indicate that constitutive expression of ASF1A favors the maintenance of pluripotency, indicating its role in pluripotency acquisition.

### Example 2: ASF1A Expression Enhances Cellular Dedifferentiation

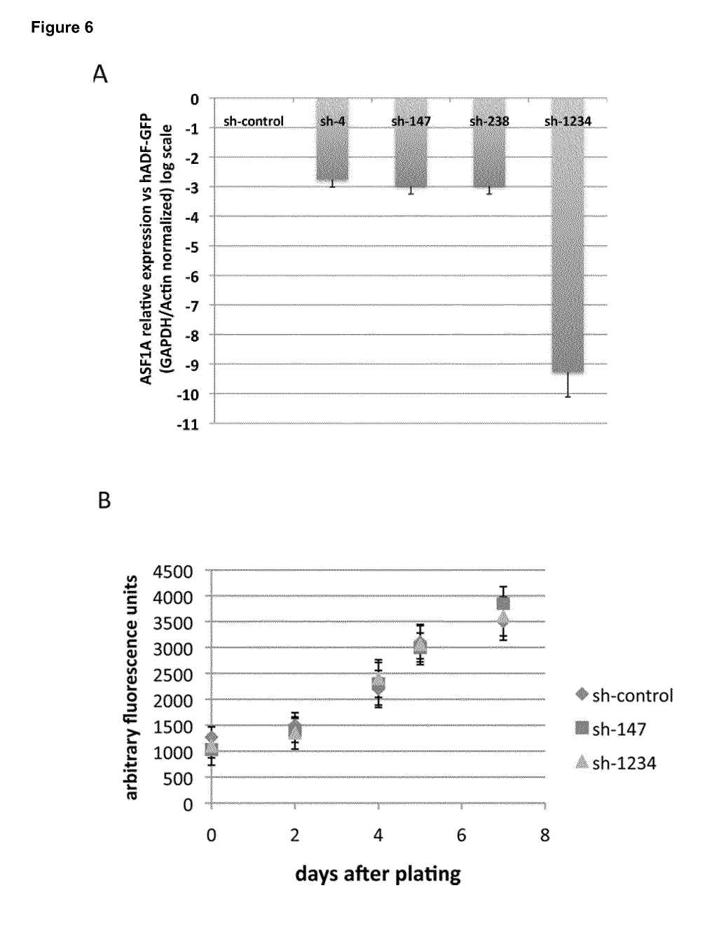

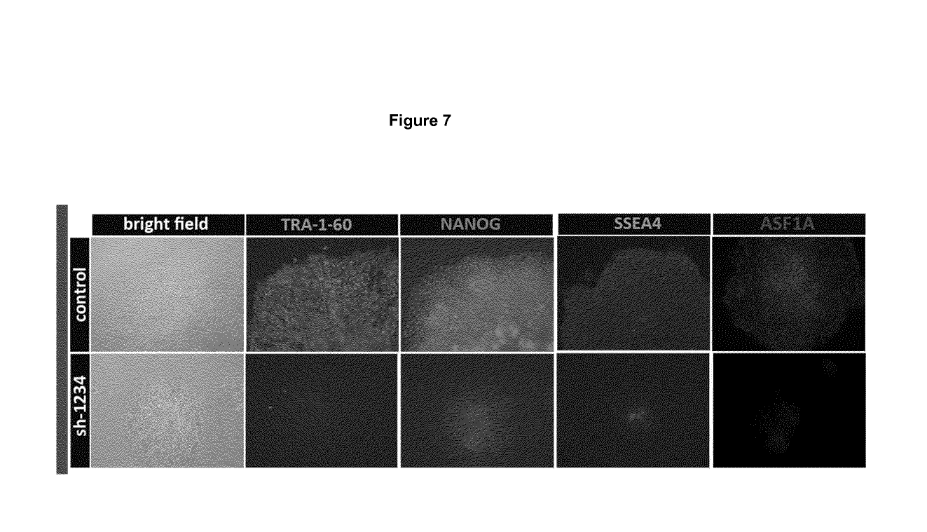

To determine whether ASF1A expression is required for cellular dedifferentiation into induced pluripotent stem cells (iPSCs), ASF1A expression was blocked using shRNA (FIG. 6A) and hADF were subsequently transduced with OCT3/4, SOX2, KLF4, and c-MYC (OSKM, “the Yamanaka factors”). We used two different ASF1A shRNA (ASF1A shRNA-147 and ASF1A shRNA-1234) or control shRNA. Downregulation of ASF1A did not alter cell proliferation rates of hADF (FIG. 6B). When shRNA-147 was used, a significant decrease in the number of TRA-1-60+ reprogrammed iPSCs colonies was observed. When the more efficient of the two shRNAs (shRNA-1234) was used, the appearance of TRA-1-60+ reprogrammed iPSC colonies was completely blocked (FIG. 2B). When the same ASF1A-shRNA vector was used to downregulate ASF1A expression in hESC-H9, a reduction in the expression of pluripotency markers was observed (FIG. 2C) along with a change in colony morphology (FIG. 7) was observed as ASF1A decreased. These experiments show that ASF1A expression is required for pluripotency maintenance and for reprogramming hADFs into iPSCs.

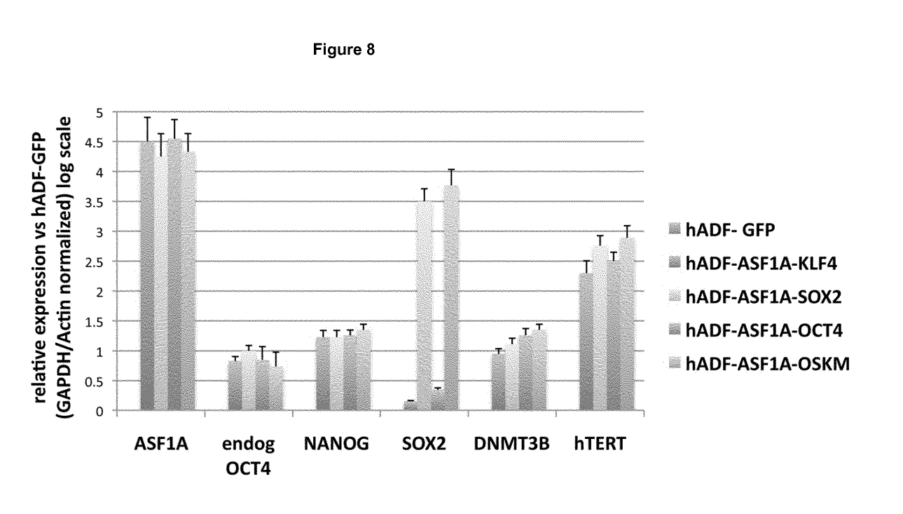

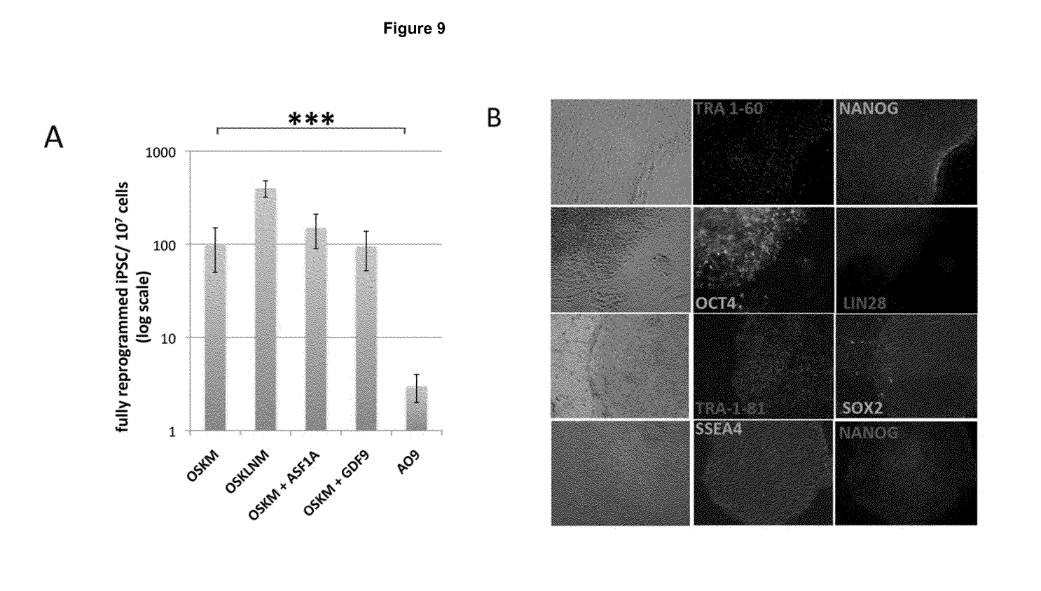

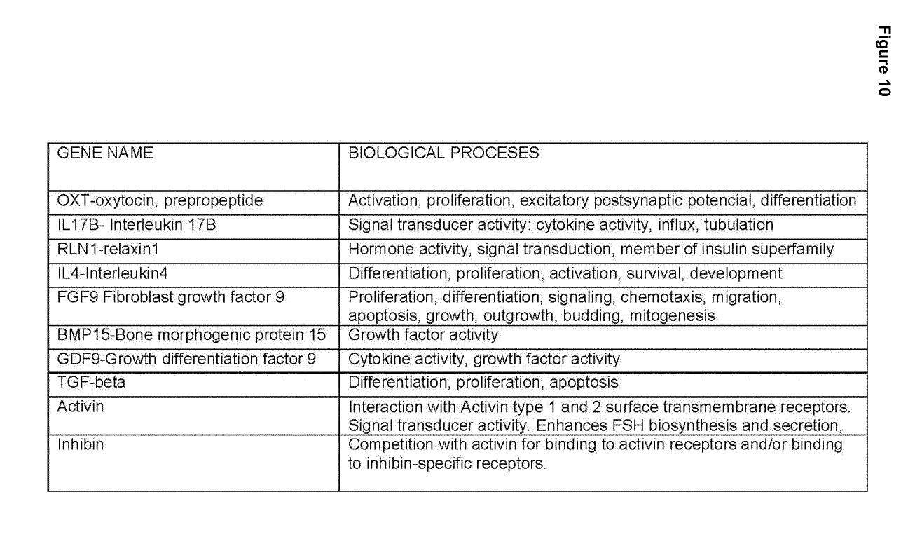

To further analyze the role of ASF1A in the pluripotent state of a cell and its interaction with the master reprogramming genes, ASF1A was overexpressed along with the Yamanaka factors individually (OCT3/4, SOX2, and KLF4) and together (OSKM). One week after transduction, no difference in the pluripotent gene expression pattern among the different combinations was observed (FIG. 8). Three to four weeks after transfection, however, the combination of ASF1A and OCT3/4 alone generated pre-iPSC-like colonies. Dermal fibroblasts transduced with OSKM plus ASF1A resulted in an increase in TRA-1-60+ iPSC-like colonies (FIG. 9A) over fibroblasts transduced with OSKM alone.

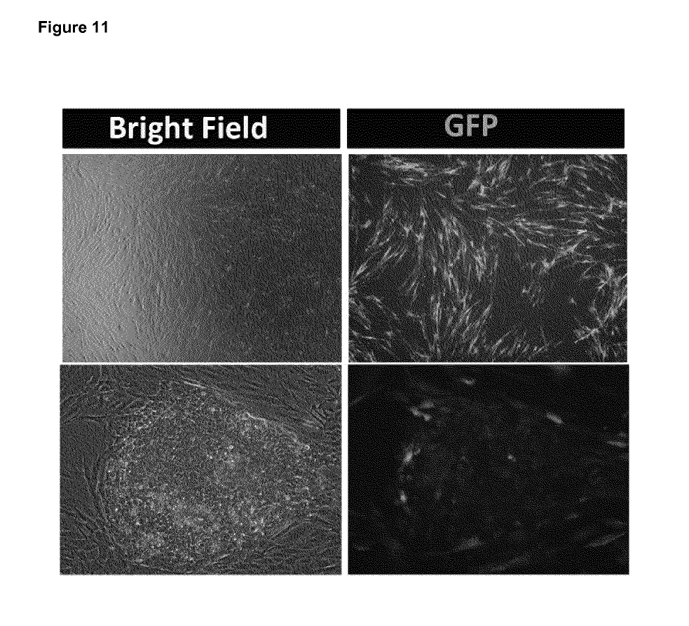

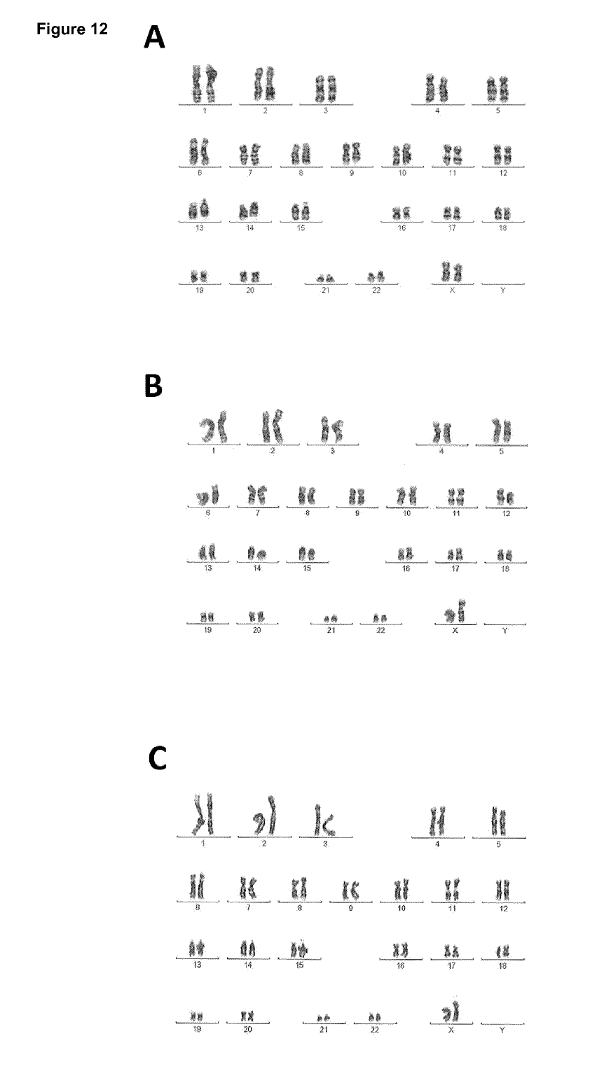

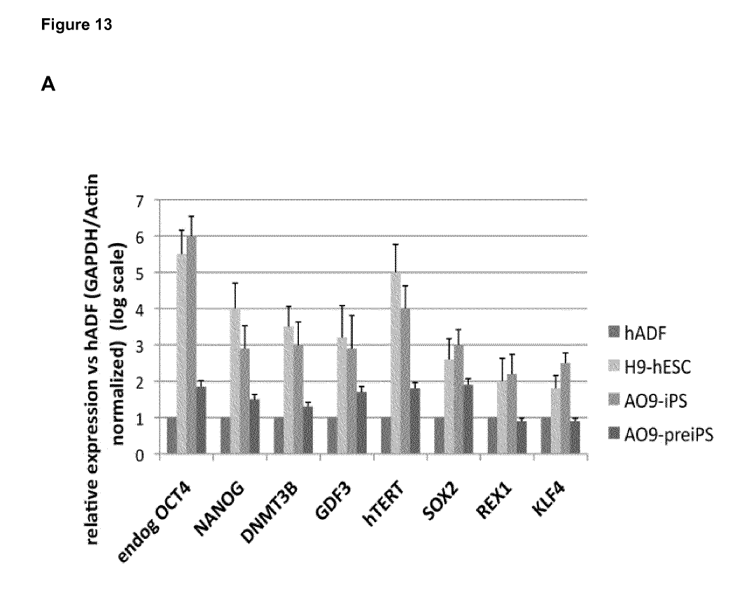

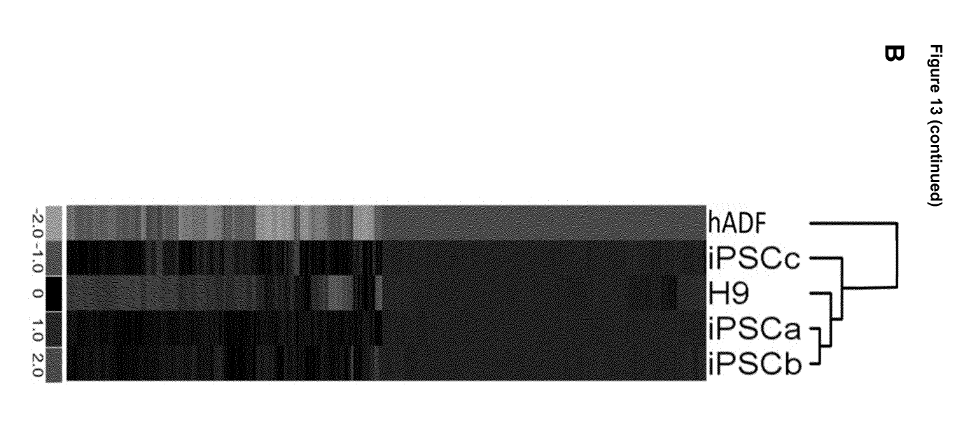

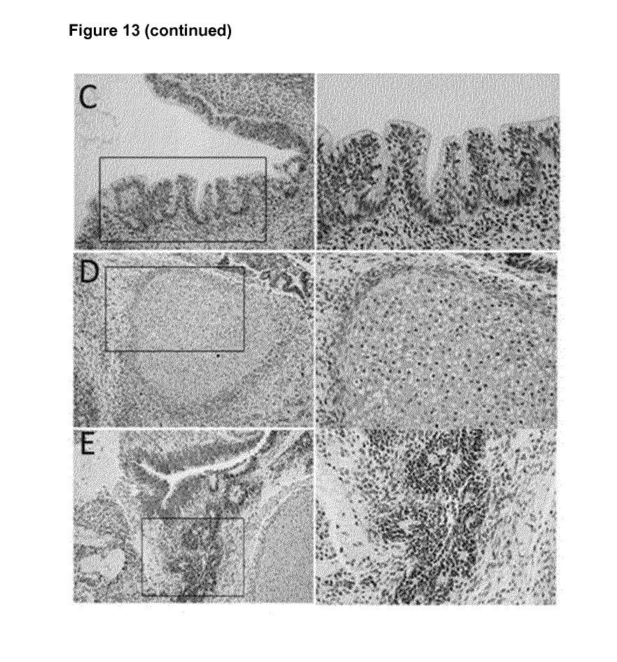

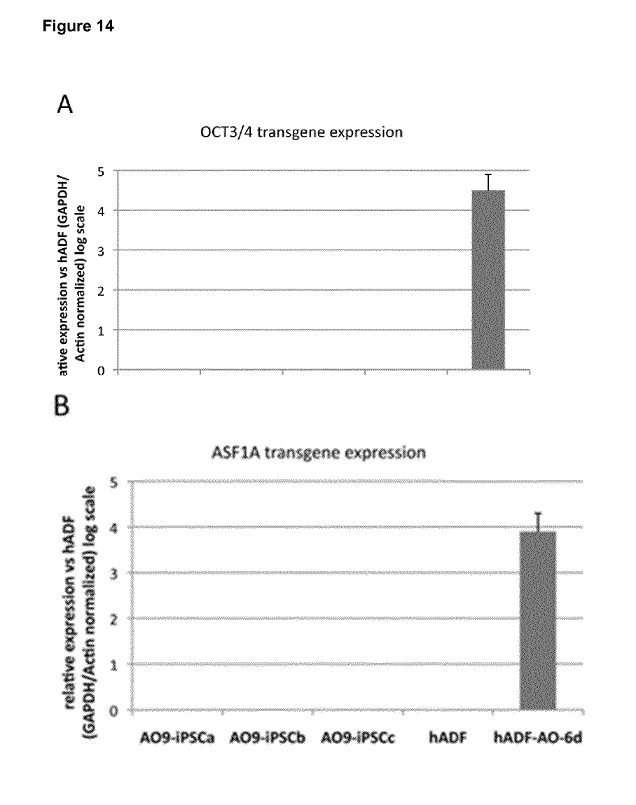

We examined whether other oocyte factors could be necessary to efficiently achieve complete iPSCs formation. Paracrine factors secreted by the oocyte itself, which are known to have well-described signaling pathways in the mammalian MII oocyte, were examined. Seven different ligands in combination with the overexpression of ASF1A and OCT4 were tested (FIG. 10). Addition of GDF9 48 hours after ASF1A/OCT4 transduction resulted in the generation of colonies with typical iPSC morphology (5±2×10⁻⁷% of transduced cells; FIGS. 9A, 9B and 11). Overexpression of OCT3/4 alone or in the presence of GDF9 did not produce any reprogrammed colonies. ASF1A-OCT4-GDF9 (AO9)-derived colonies were fully reprogrammed, showing a normal karyotype (FIG. 12) and expressing standard stem cell markers after culturing for six to ten passages (FIGS. 13A, 9B), and showing a gene expression profile similar to hESCs (FIG. 13B). No detectable expression of exogenous ASF1A/OCT4 from the retroviral vectors in the AO9-iPSC clones was found 65 days after transduction (FIG. 14).

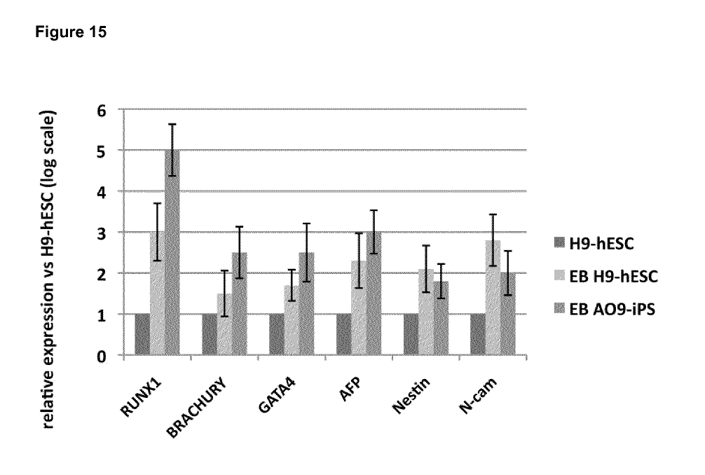

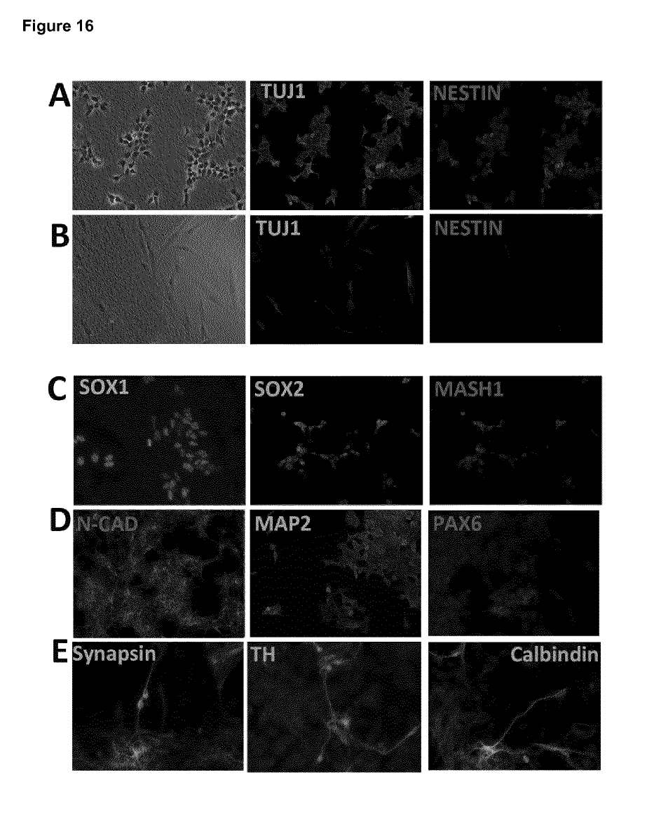

When induced to differentiate in vitro, fully reprogrammed AO9-iPSCs can form ecto-, endo-, and mesoderm cell lineages (FIGS. 15 and 16). Injection of AO9-iPSC lines into immunodeficient mice formed mature teratomas that had intestinal epithelium (endoderm), cartilage (mesoderm) and neural epithelium (ectoderm). (FIGS. 13C-E)

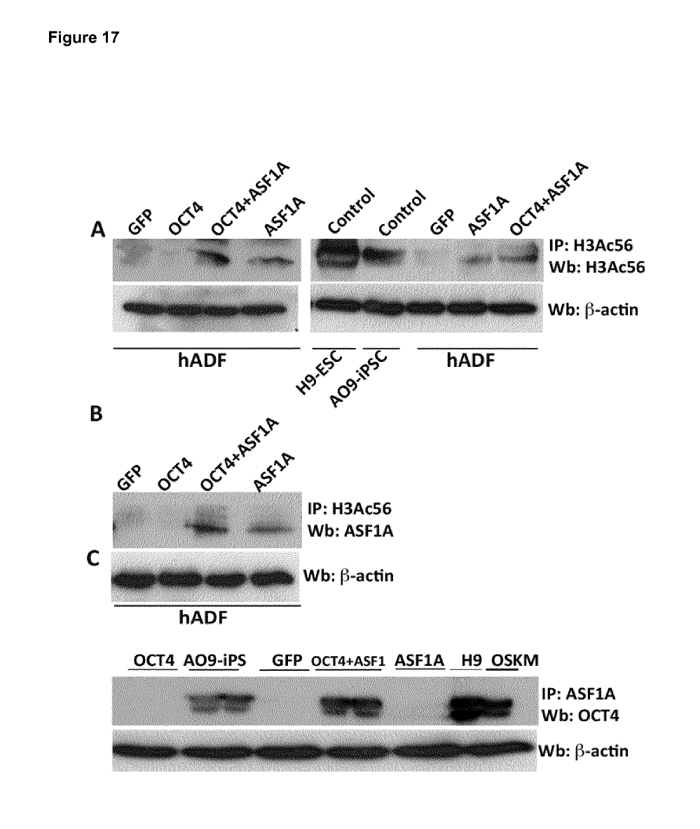

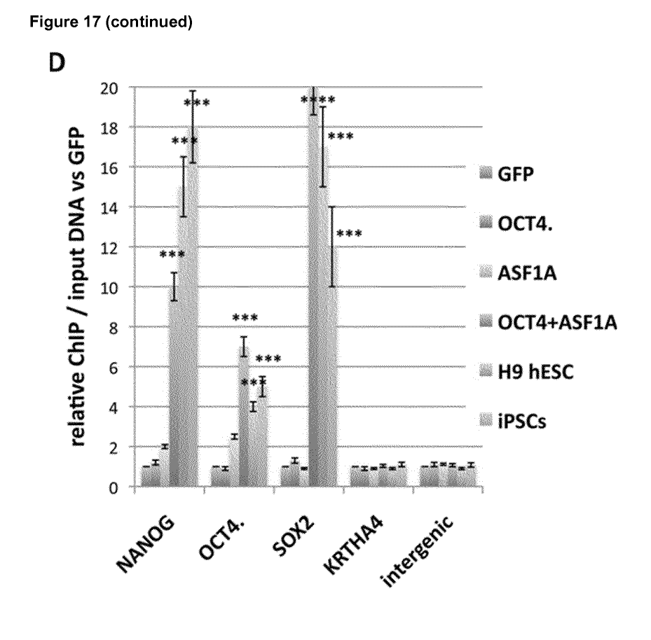

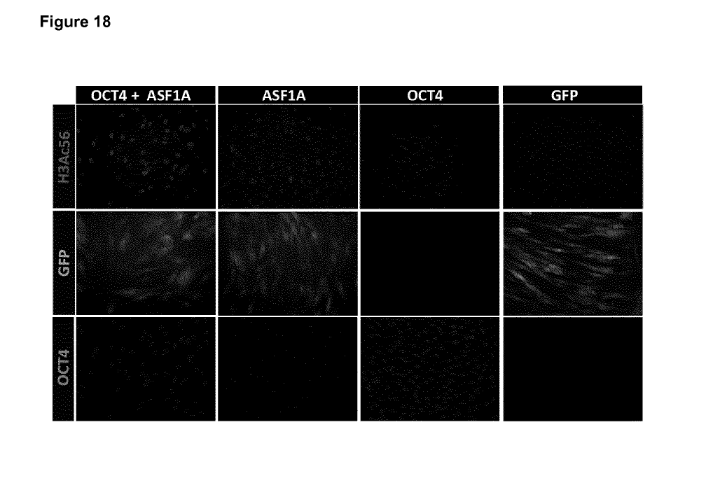

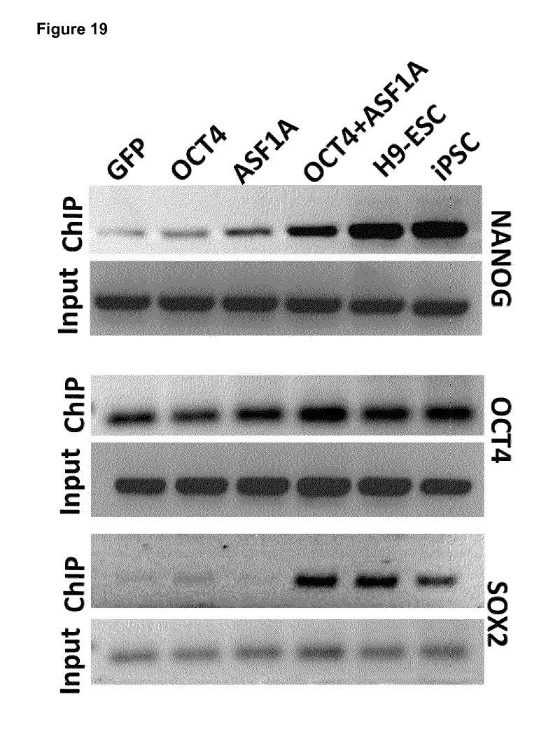

At the epigenetic level, overexpression of ASF1A on human dermal fibroblasts increased H3K56Ac significantly, and the acetylation was even higher when OCT3/4 was coexpressed in the same cells (FIGS. 17A and 18). The interaction between ASF1A and H3K56Ac was confirmed in hADFs, hiPSCs, and hESCs (FIG. 17B). When hADF were examined 72 hours after the overexpression of the ASF1A-OCT3/4 factors and it was observed that these two factors co-immunoprecipitate (FIG. 17C). ChIP analysis confirmed that H3K56Ac is found in regulatory regions of NANOG, OCT4, and SOX2 after overexpression of ASF1A (FIGS. 17D and 19). These results indicate that ASF1 and OCT4 are capable of activating genes at the core of the pluripotency regulatory network, at least in part through the acetylation of H3K56.

### Example 3: Gene Expression in ASF1A Transduced Cells

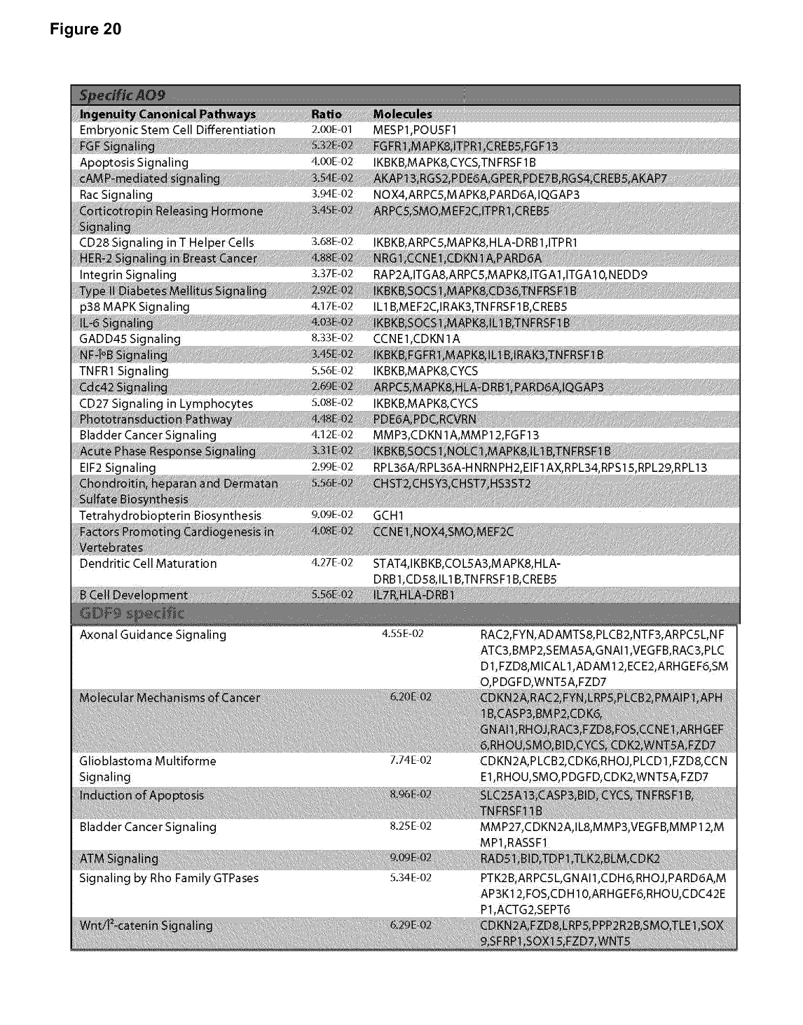

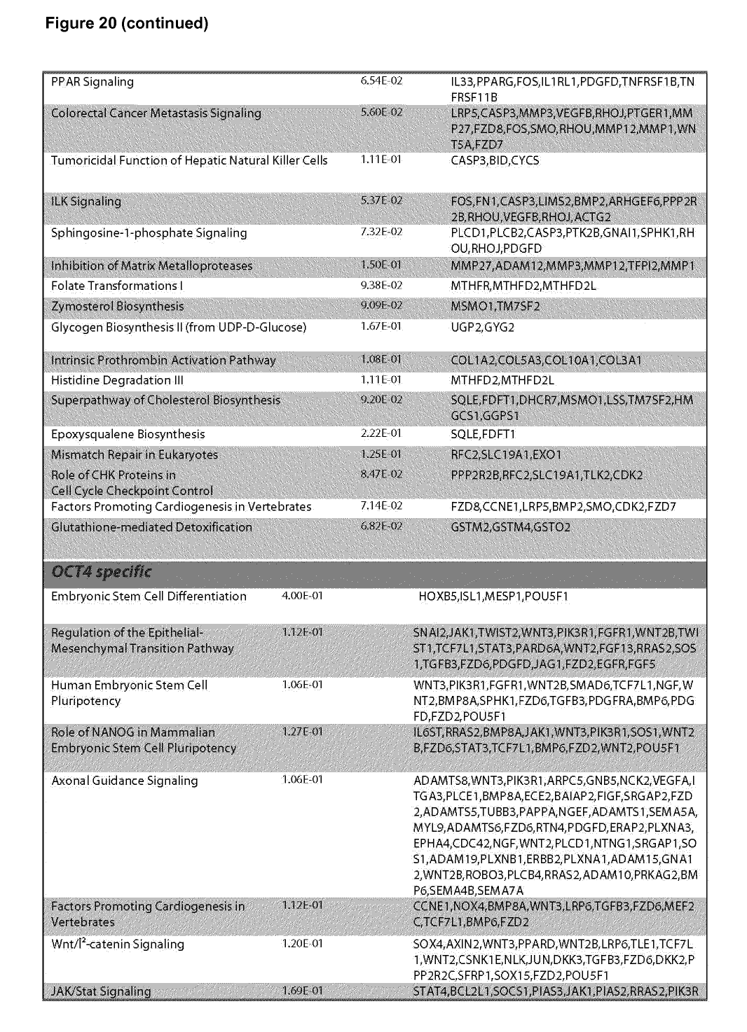

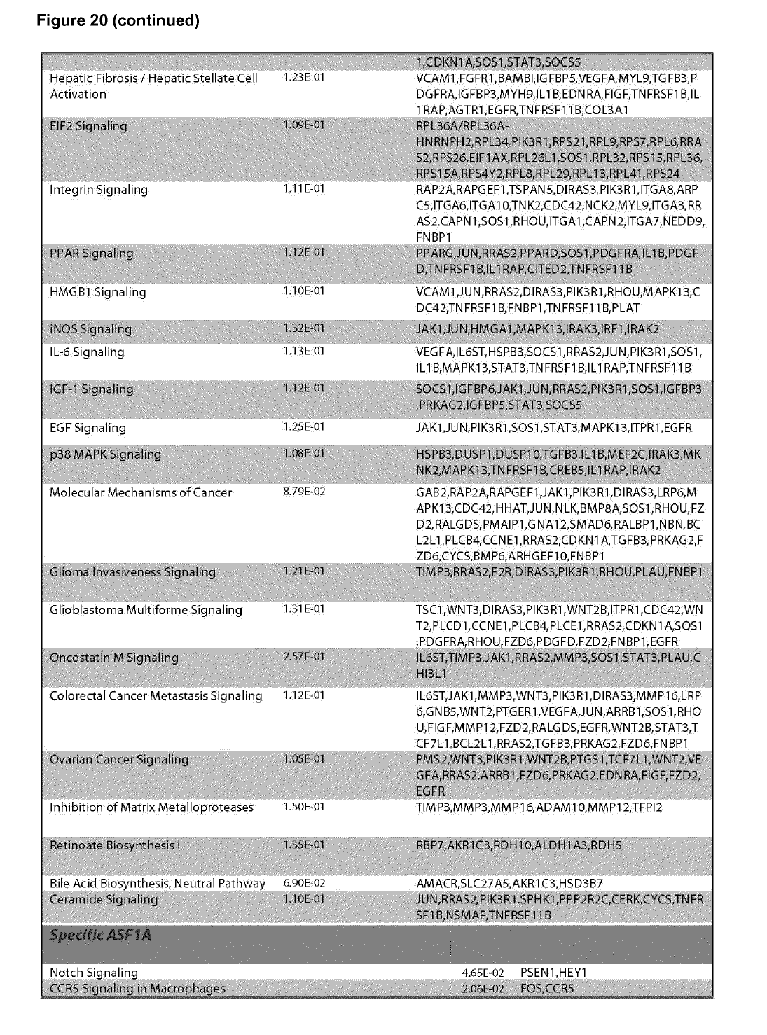

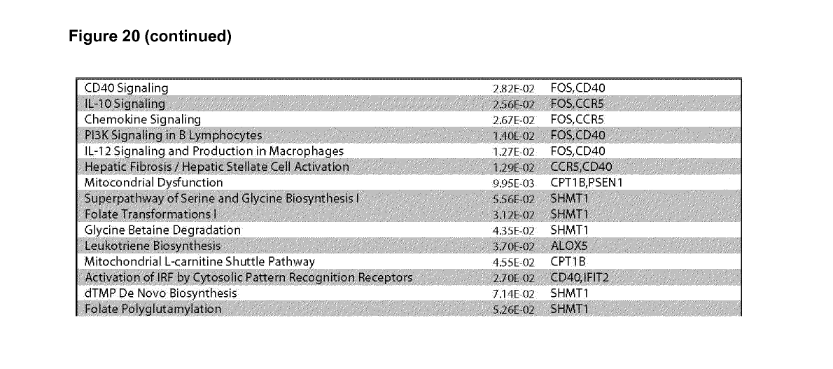

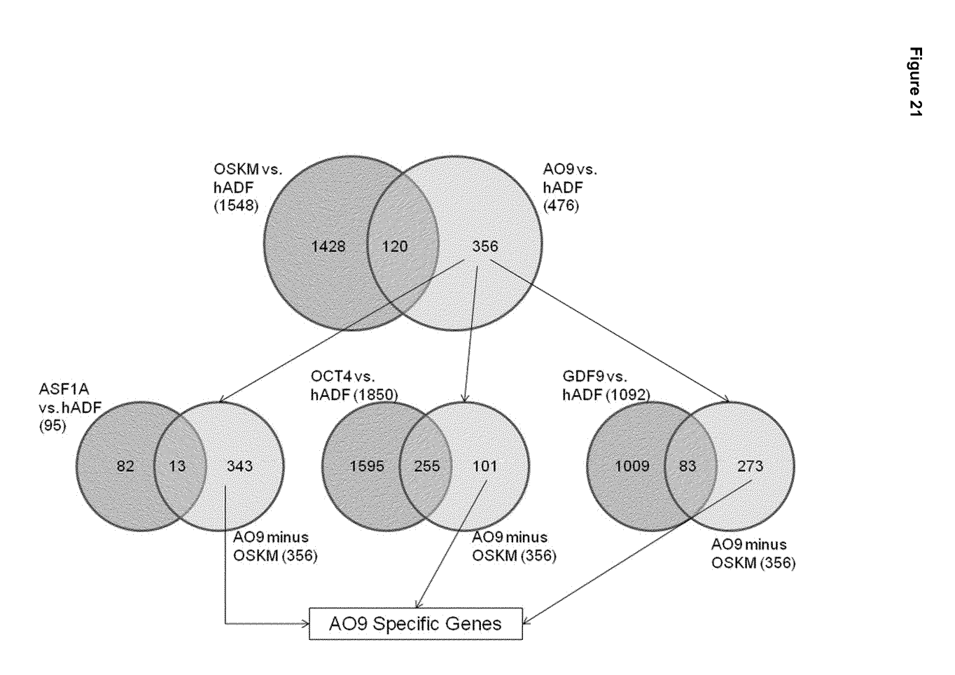

To elucidate the signaling pathways involved in the AO9 reprogramming process, global gene expression profiles of human dermal fibroblasts were analyzed 48 hours after exposing cells to the individual factors both alone and in combination (i.e., overexpression of ASF1A or OCT4, or exposure to GDF9, or AO9). Using Ingenuity Pathway Analysis, (Redwood City, Calif.) (FIG. 20), it was found that AO9 overexpression regulates, among other signaling pathways, p38 and IL-6 signaling. These are also regulated after OCT4 single factor overexpression, and are crucial to the reprogramming process. Other signaling pathways identified in this analysis include CD28, TNFR1, CDC42, CD27, and NF-kB.

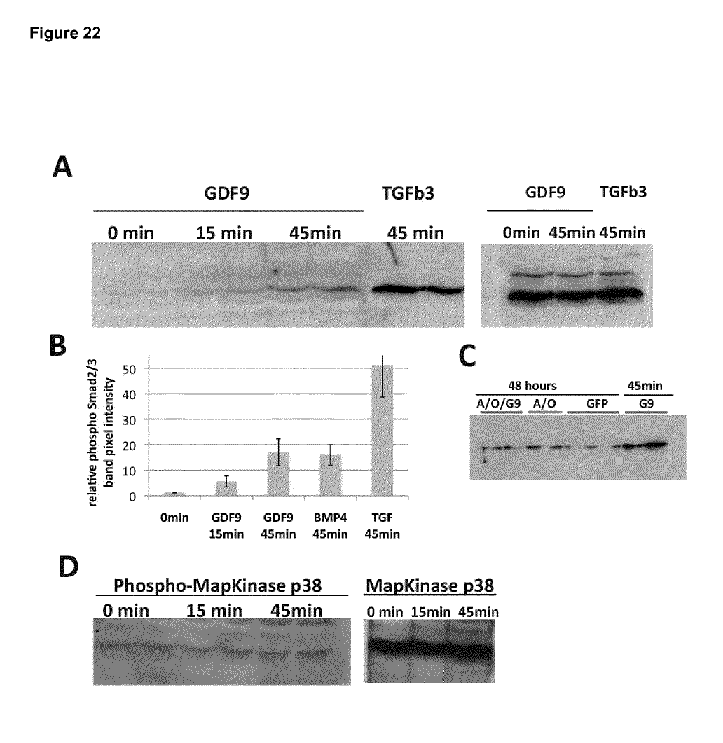

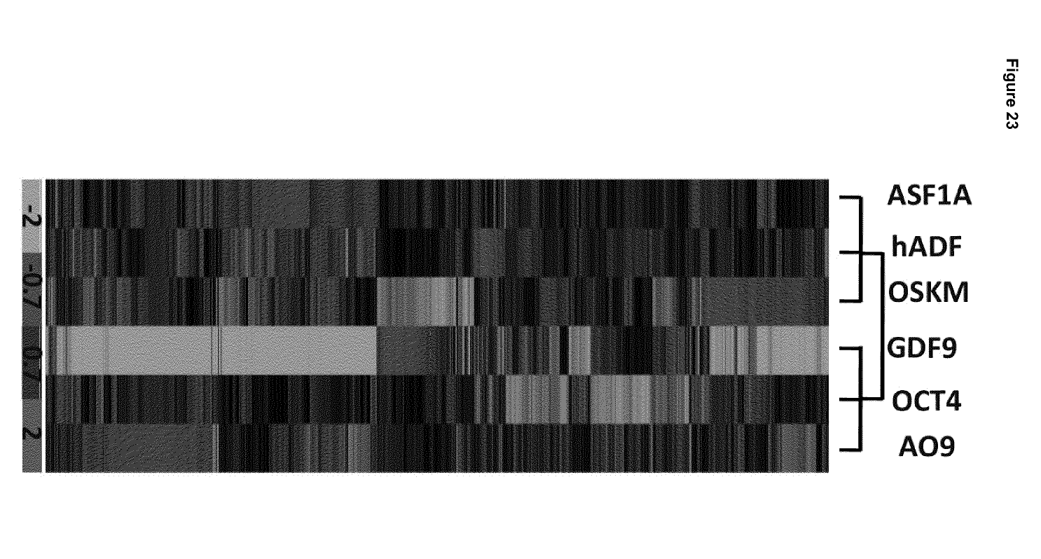

The above analysis revealed that GDF9 activates R-SMADs 2/3 phosphorylation on human dermal fibroblasts, but not ERK1/2 (FIGS. 21 and 22). GDF9 exhibited a different function from its already described role in ovarian folliculogenesis. Other pathways activated by exposing hADFs to GDF9, that are crucial for cell reprogramming to occur, include apoptosis, Wnt/β catenin signaling, cell transformation, and PPAR signaling.

Global gene expression profiles of human dermal fibroblasts were analyzed 48 hours after exposing cells to the individual factors either alone or in combination. As a control, the same cells transduced with either GFP alone or with OSKM were used. Comparing the AO9 cell treatment to the GFP control, 476 differentially expressed genes were observed, some of which did not exhibit significant alterations in expression after OSKM overexpression.

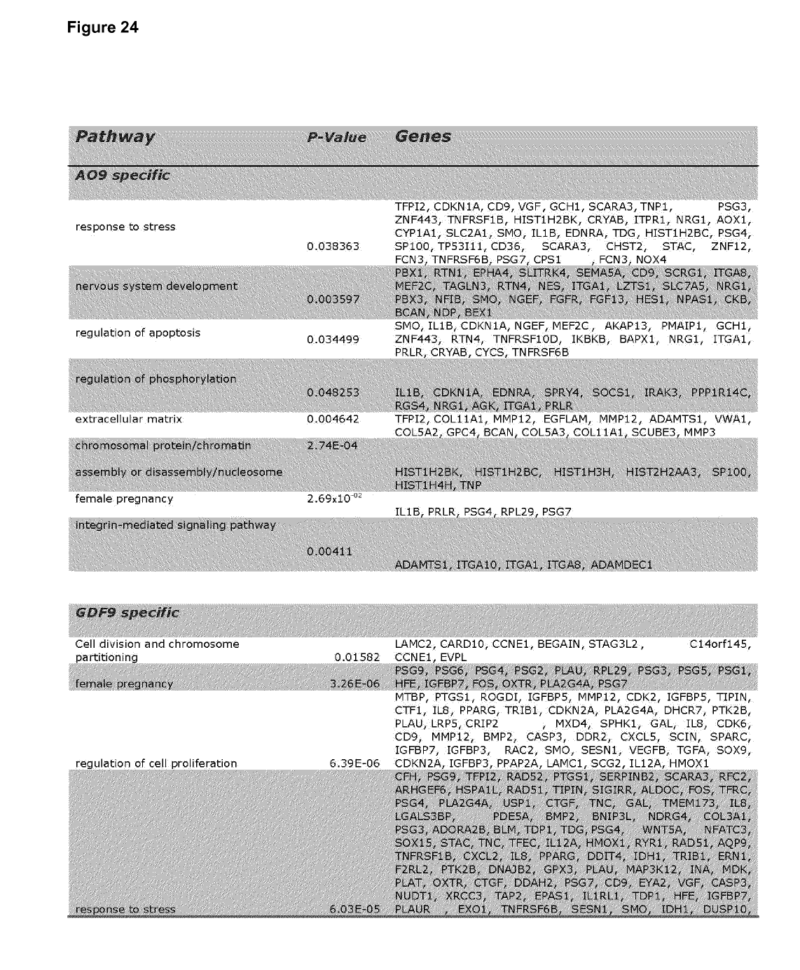

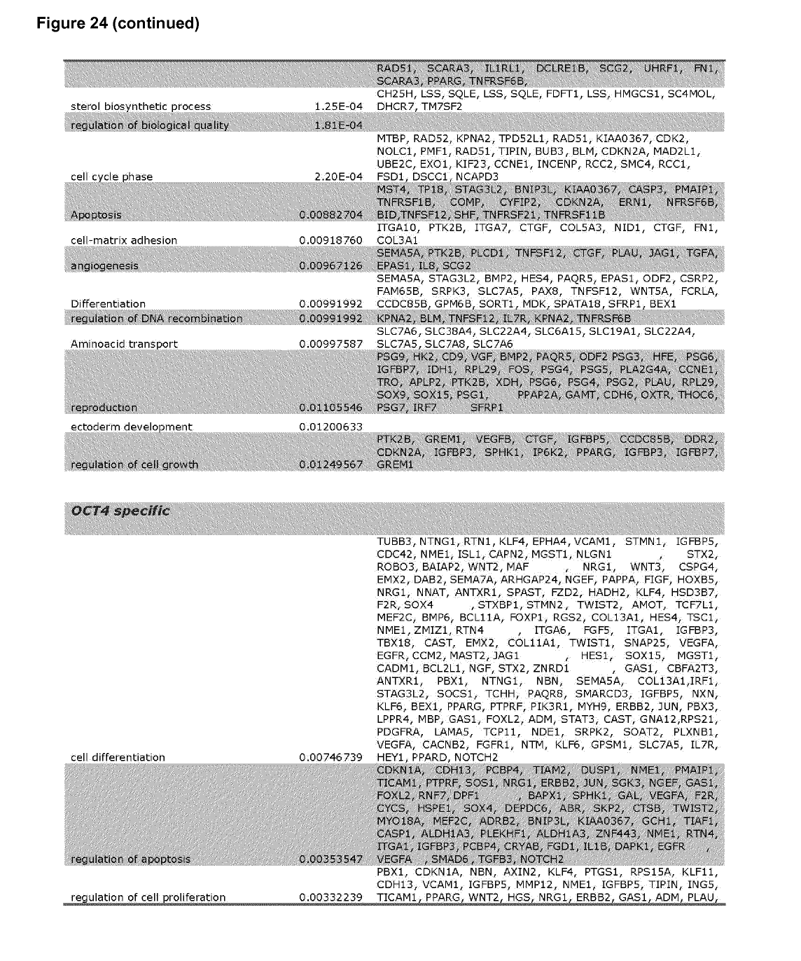

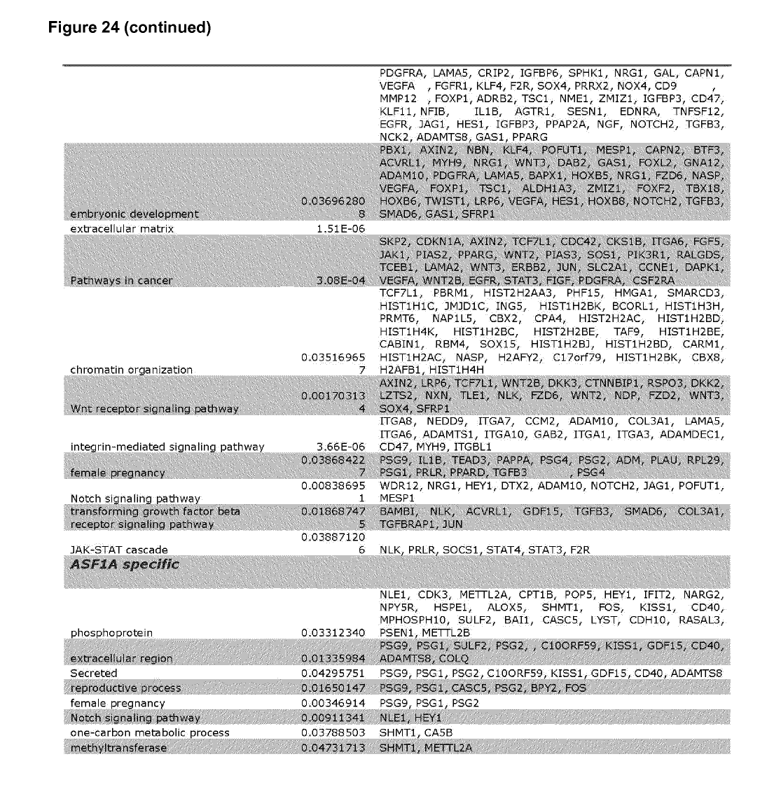

Similarly, genes that were specifically changed after each single-factor overexpression and yet, at the same time, differed from the ones that changed after AO9 overexpression were observed. Of note, 356 genes were found that were differentially expressed in AO9 but not in OSKM samples when compared to the GFP control (FIG. 23). This list of genes was compared to the genes that have are up- or down-regulated following the induction of the single factors ASF1A, OCT4, or exposure to GDF9. A set of 349 genes were found that had been specifically deregulated due to the combined induction of the three factors that did not exhibit a significant change either after OSKM induction or when these three factors are induced in singularity (FIG. 21). Functional analysis of these genes identified GO categories related to response to stress, apoptosis, chromatin organization, extracellular matrix modification, female pregnancy, and integrin signaling (FIG. 24).

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. An in vitro method of inducing the dedifferentiation of a somatic cell, the method comprising:
a) inducing expression of ASF1A in a somatic cell; and
b) culturing the cell.

2. The method of claim 1, wherein the method further comprises inducing expression of OCT3/4, NANOG, SOX2 or DNMT3B in the cell prior to step b).

3. The method of claim 1, wherein the method further comprises inducing expression of OCT3/4 in the cell prior to step b).

4. The method of claim 1, wherein the method further comprises inducing expression of OCT3/4, NANOG, SOX2 and DNMT3B in the cell prior to step b).

5. The method of any of claims 1 to 4, wherein the expression of ASF1A is induced in step a) by contacting the somatic cell with a ASF1A expression vector.

6. The method of claim 5, wherein the ASF1A expression vector is retroviral vector.

7. The method of claim 6, wherein the ASF1A expression vector is self-inactivating retroviral vector.

8. The method of any of claims 1 to 7, wherein the somatic cell is a human cell.

9. The method of claim 8, wherein the somatic cell is a human fibroblast cell.

10. The method of any of claims 1 to 9, wherein step b) comprises culturing the cell in human ES cell medium.

11. The method of any one of the preceding claims, further comprising the step of contacting the cell with GDF9.

12. A method of making an induced pluripotent stem (iPS) cell from a somatic cell, the method comprising:
a) inducing expression of ASF1A and OCT3/4 in a somatic cell; and
b) contacting the cell with GDF9 and culturing the cell under conditions whereby the somatic cell becomes an iPS cell.

13. The method of claim 12, wherein the method further comprises inducing expression of NANOG, SOX2 or DNMT3B in the cell prior to step b).

14. The method of claim 12, wherein the method further comprises inducing expression of NANOG, SOX2 and DNMT3B in the cell prior to step b).

15. The method of any of claims 12 to 14, wherein the expression of ASF1A is induced in step a) by contacting the somatic cell with a ASF1A expression vector.

16. The method of claim 15, wherein the ASF1A expression vector is retroviral vector.

17. The method of claim 16, wherein the ASF1A expression vector is self-inactivating retroviral vector.

18. The method of any of claims 12 to 17, wherein the somatic cell is a human cell.

19. The method of claim 18, wherein the somatic cell is a human fibroblast cell.

20. The method of any of claims 12 to 19, wherein step b) comprises culturing the cell in human ES cell medium.

21. A dedifferentiated somatic cell obtained or obtainable according to the method of any of claims 1 to 11.

22. An induced pluripotent stem (iPS) cell obtained or obtainable according to the method of any of claims 12 to 20.

23. A dedifferentiated somatic cell characterized by an increased expression and/or activity of ASF1A in comparison to a somatic cell that has not been contacted with an agent capable of increasing the expression and/or activity of ASF1A.

24. A dedifferentiated somatic cell characterized by an increased expression and/or activity of ASF1A and OCT3/4 in comparison to a somatic cell that has not been contacted with one or more agents capable of increasing the expression and/or activity of ASF1A and OCT3/4.

25. An induced pluripotent stem (iPS) cell characterized by an increased expression and/or activity of ASF1A in comparison to a somatic cell that has not been contacted with an agent capable of increasing the expression and/or activity of ASF1A, wherein said iPS cell is further characterized by not having an induced expression of oncogenes c-MYC or KLF4.

26. An induced pluripotent stem (iPS) cell characterized by an increased expression and/or activity of ASF1A and OCT3/4 in comparison to a somatic cell that has not been contacted with one or more agents capable of increasing the expression and/or activity of ASF1A and OCT3/4, wherein said iPS cell is further characterized by not having an induced expression of oncogenes c-MYC or KLF4.

27. A cell population comprising a cell as defined in any of claims 21 to 26.

28. A substantially pure population comprising a cell as defined in any of claims 21 to 26, wherein the term substantially pure is understood as the population comprising a percentage of the cell as defined in any of claims 21 to 26 of at least 80%, preferably 85%, more preferably 90%, 95%, 96%, 97%, 98%, 99% over the total number of cells of the population.

29. A pharmaceutical composition comprising a cell as defined in any of claims 21 to 26 or the cell population as defined in any of claims 27 or 28, further comprising a pharmaceutically acceptable carrier.

30. The cell as defined in any of claims 21 to 26, the cell population as defined in any of claims 27 or 28, or the pharmaceutical composition as defined in claim 29, for use in therapy.

31. The cell as defined in any of claims 21 to 26 or the cell population as defined in any of claims 27 or 28, or the pharmaceutical composition as defined in claim 29, for use in a cell therapy method, in particular for use in tissue and/or organ repair and regeneration.
